(19) United States
(12) Patent Application Publication
(10) Pub. No.: US 2006/0057213 A1
(43) Pub. Date: Mar. 16, 2006

(54) Drug delivery particles and methods of treating particles to improve their drug delivery capabilities

(76) Inventors: Hassane El Larhrib, Liverpool (GB); Joseph Okpala, Liverpool (GB)

Correspondence Address: DENNISON, SCHULTZ, DOUGHERTY & MACDONALD, 1727 KING STREET, SUITE 105, ALEXANDRIA, VA, 22314, US

(21) Appl. No.: 10/538,490

(22) PCT Filed: Dec. 10, 2003
(86) PCT No.: PCT/GB03/05353

§ 371 Date: Jun. 9, 2005

(30) Foreign Application Priority Data
Dec. 11, 2002 (GB) 0228826.4

(51) Int. Cl. A61K 9/14

(52) U.S. Cl. 424/489

(57) ABSTRACT

A first embodiment of the invention provides a method of engineering changes in the morphological, chemical or physical features of a particle, to promote, for example, the formation of hairs and pores on the surface of the particle. The presence of these engineered features facilitating the delivery of agents to a target region, such as beclomethasone to the alveoli of the lungs. Although the particle acts to deliver desired agents to a target region, the particle itself may also be an agent. The invention also provides particles specifically engineered by the above method so as to produce carrier particles suitable for particular tasks.

### FIELD OF THE INVENTION

The invention relates generally to the field of selectively modifying the morphological, physical and chemical features (architecturing) of particles to improve the delivery characteristics of the particles. In particular, the invention relates to the production and/or modification of particles for delivery via inhalation.

### BACKGROUND OF TE INVENTION

Drugs for treating respiratory and nasal disorders are frequently administered through the mouth or nose as fine particles incorporated into a formulation. The particles between 1-5 μm are regarded as respirable, i.e. capable of penetrating into the lungs. Particles for nasal delivery are generally, slightly larger (1-10 μm). Conventional dry powder nasal formulations are often the same as, or are slight modifications of inhalation formulations of the same drug. It is obvious that there is an overlap in the particle size distribution, hence formulations intended for nasal delivery will contain particles less than 5 μm. Such standard nasal formulations are inefficient for a number of reasons. Firstly, particles of 5-10 μm are more likely to deposit in the nose, whereas, the particles below 5 μm are more likely to pass the nose and reach the pharynx, trachea and lung and are considered wasted. Because of the wastage, the portion of the drug remaining in the nose is insufficient to treat the nasal condition, furthermore, the wasted particles are likely to cause unwanted side effects due to their deposition beyond the nasal cavity. In a similar manner, deposition of drugs intended for the lungs in the oropharyngeal cavity may gain access to the systemic circulation causing unwanted side effects. These two examples show the importance of controlling not only the particle size but also particle size distribution. Furthermore, it is also important to control other particle morphological features (particle shape, particle density, surface texture of the particles to name a few examples) in order to ensure efficient therapy, while minimising any unwanted side effects.

Techniques known in the art to obtain particles of 1-5 μm and 1-10 μm are milling, crystallisation, spray-drying and supercritical fluid to name a few common examples, however these techniques are fraught with problems as briefly outlined below.

The milling process is undesirable for several reasons. It has the potential to change more than the particle size of the feed material. The heat generated during milling reduces crystallinity and stability and causes chemical deposition of thermally labile molecules. Additionally, during micronisation brittle materials will tend to fracture during inter-particle collisions while ductile materials can not be milled as they deform plastically rather than fracturin. In addition, the milled powder is highly cohesive and thus very difficult to mix due to poor and incomplete dispersion of agglomerates into their single particles. Milling also generates a significant fraction of unwanted under-size particles that must be removed for the reasons outlined above and are thus considered wasted making the milling process uneconomic. Additionally milling does not give the user control over particle density, particle shape and particle surface texture. Furthermore, the milling process exposes the personnel to the hazardous effect of the fine dust coupled with high product loss.

Particles of this size range (1-5 μm, 1-10 μm) are extremely difficult to achieve by crystallisation. The crystallisation process requires considerable time and energy resources and defines such economical issues as efficiency of solvent recycling, separation of waste (impurities) and consumption of raw materials. It is acknowledged that minor changes in crystallisation conditions, for example supersaturation, temperature, impurity or cooling rate can produce significant changes in the crystal and powder properties notably, particle size, shape, purity and defect structure followed by less pronounced but significant variations in thermodynamic and mechanical properties. These effects have been recognised as the major batch to batch and source variation problems leading to inconsistencies of the final product.

Spray drying has been seen as an alternative technique to micronisation as the shape of the particle is spherical and can be easily controlled whilst producing particles with a narrow size distribution. However, the material formed contains various degrees of amorphous regions. Such regions are often more sensitive to external conditions e.g. moisture, thus making the particles more susceptible to chemical degradation. This technique is impracticable for heat-sensitive materials and suffers from low product yield. Furthermore, the particles produced are always cohesive and have poor flow and hence cannot be realistically aerosolised (Kawashima, Y et al., (1998), Effect of surface morphology of carrier lactose on dry powder inhalation property of pranlukast hydrate, International Journal of Pharmaceutics, 172, 179-188).

Particles produced using supercritical fluids has been proposed as another alternative technique to micronisation, unfortunately, this technique does not enable control over particle shape, particle density and is unsuitable for large scale industrial production. In addition, supercritically processed particles are acicular (i.e. not spherical) and thus more difficult to mix, having prolonged mixing times due to their tendency to agglomerate and segregate compared to spherical particles (rain, D. Pharmaceutical aspects of mixing solids, Pharm. J. 1960, 185, 129-134). Additionally acicular particles have poor flow properties (Staniforth, J. N., Powder Flow, In Pharmaceutics, The Science of Dosage Form Design; Aulton, M. E., Ed; Churchill Livingstone; London, 1988; 600-628).

Important aspects in the use of small particles as obtained by the above techniques, for inhalation (for example), are their instability, cohesiveness and poor flow properties. Despite these aspects, particles produced by the above techniques are in reality currently used in different pharmaceutical areas to the detriment of the formulation and effective drug delivery. This also severely limits the use of these particles, alone, or as carrier particles for inhalation.

In order to rectify one of the aspects as mentioned above, in this case instability, Patent WO 95/05805 describes the rearrangement and conditioning of fine-grained substance by treatment with a water vapour phase to produce a stable crystalline powder. Here, however, the particle size and aerodynamic properties of the particles were maintained as before conditioning. Thus, if the powder before treatment contains under-size particles the latter will still remain in the final treated product and this, as discussed previously, may cause side-effects. Despite ameliorating one of the above aspects, Patent WO 95/05805 did not improve the aerodynamic properties, in particular the aerodynamic diameter of the particles.

The aerodynamic diameter is a major parameter dictating the deposition of inhaled particles in different regions of the airways. The aerodynamic diameter is given by the equation:
dₐ=d_{g}(ρₚ/ρ₀χ)^{0.5}
where dₐ is aerodynamic diameter of the particle;
d_{g} is geometric diameter of the particle; ρₚ is the particle density,
ρ₀ is a reference density of 1 g/cm³; and χ is the dynamic shape factor, which is 1 for a sphere.

It is evident from the above equation that there is a direct relationship between aerodynamic diameter and both particle density and particle geometric diameter. For example, a perfectly spherical 10 μm particle with a density of 1.5 g/cm³ has an aerodynamic diameter of 12.25 μm. For the same perfectly spherical 10 μm particle whose density was reduced to 0.01 g/cm³ has an aerodynamic diameter of 1 μM. It is clear that dₐ can be maintained or controlled by altering ρₚ or altering d_{g} or altering both ρₚ and d_{g}. From the literature, it is clear that particles with aerodynamic diameter >10 μm will not enter the tracheobronchial tree, whilst particles with the same geometric diameter and low particle density giving a low aerodynamic diameter (1 μm) will not only penetrate the tracheobronchial tree, but also reach the alveolar space (Sandra Suarez and Anthony. J. Hickey. 2000. Drug Properties Affecting Aerosol Behaviour. Respiratory Care, 45 (6) 652-666).

It has also been shown that particles with small aerodynamic diameter are more attractive for inhalation therapy (Rita Vanbever, Jeffrey D. Mintzes, Jue Wang, Jacquelyn Nice, Donghao Chen, Richard Batycky, Robert Langer, and David A. Edwards. Formulation and physical characterisation of large porous particles for inhalation, Pharmaceutical research, vol. 16, NO. 11, 1999), because they deposit in the alveoli space where there is good contact with the blood stream. It is clear that the particles conditioned described according to the process in WO 95/05805 are not optimal for delivery to the lung as it did not bring any improvements in the aerodynamic properties (i.e. there were no changes in both particle size and particle density). It is crucial, for inhalation, that the aerodynamic properties should be improved in order to increase the medical value of inhaled drug particles.

Improvements have been obtained in drug delivery to the lower airways by lowering the density of the drug particles themselves, which in turn reduces the aerodynamic diameter, [See references: Edwards et al, Science, 276 (5320), 1868-1871, 199; Vanbever et al., 1999; Bosquillon et al, 2001; Ben-jebra et al 19991]. This reduction in aerodynamic diameter enables easy aerosilization and dispersion within the air stream, thus allowing more drug particles to be become deposited in the lower airways. U.S. Pat. No. 6,284,282 discloses a process of applying the principle of producing larger particle of low density so as to achieve drug lung depositions in excess of 40% of the administered dose using carrier/drug ratios of 10 to 1 w/w. This is strikingly superior to the conventional formulations (i.e. carrier/micronised drug). The engineered, low density drug particle of U.S. Pat. No. 6,284,282, aerosolize easily from the inhaler device; as a result less carrier is required. The improvements made come at a price in that typically 4-5% of the drug was included in a multi-component system. Such complex systems increase the possibility of physical incompatibilities between components and may be undesirable in terms of patient acceptability. In addition, these complex systems will be slow release in nature as a result of the use of polymeric matrix and lipid/waxy based excipients. This is unacceptable in treating acute respiratory conditions. Furthermore, the improved aerosolization is due to the larger size (i.e. larger d_{g}) which allows better flow of the powder. It would be desirable to further reduce the aerodynamic diameter by producing smaller particle size (i.e. reducing d_{g}) while maintaining good flow and hence aerosolization. Unfortunately reducing the particle size of spray dried material (as used in U.S. Pat. No. 6,284,282) will cause increased cohesion between particles drastically reducing the flow properties of the resultant powder.

From the above, it is clear that improvements in the drug particle properties improves overall drug deposition in the lungs. Current dry powder inhaler formulations (DPIs) use micronised drug particles, however, due to the high cohesion between drug-drug particles and poor flow properties coupled with the low therapeutic drug dose thus a carrier, usually lactose (whose particle size is greater than 60 μm), is mixed with micronised drug. The carrier particles have the following three roles for DPIs; acts as a bulking agent, improves the flow properties of the formulation and hopefully allowing easy drug detachment from its surface during inhalation.

All commercially available carriers present surface irregularities that prevent drug detachment, upon inhalation, resulting in low deposition profiles and batch-to-batch variations in drug deposition profiles is usual. Commercially available lactose carriers differ from each other (in terms of particle size, particle shape, particle density, particle surface texture and polymorphic forms) depending on the source and the method by which they were produced, which affect the deposition profile of the inhaled drug. Commercial batches of lactose obtained from the same manufacturer, though possessing the same physico-chemical and technological characteristics, exhibited substantially different behaviours on inhalation, so that they could not be regarded as equivalent (Larhrib et al, 1999, The use of different grades of lactose as a carrier for aerosolised salbutamol sulphate, Int. J. Pharm. 191, 1-14).

Conventional formulations composed of a binary blend (micronised drug to carrier, typically 1 to 67.5, w/w) only manage to achieve lung drug depositions in the range of 15% of the administered dose, [See Shekunov and York, J. of Crystal Growth, 211(2000), 122-136, Lorgstrom, L., Derom, E., Stahl, E., Wahlin-boll, E. and Pauwels, R., Am. J. Respir. Crit. Care Med, V61 153, pp 1636-1640, 1996]. A high ratio of carrier to drug is used in these current formulations not because of the diluent properties of the carrier but because it has to be added in such large concentrations to improve the flow properties of the formulation.

It also follows that, since the drug is always generally mixed with a carrier and the carrier is present at a much higher concentration than the drug (1:67.5, w/w, drug:carrier, is common) in the formulation, improvements in the properties of the carrier should also improve drug deposition.

Currently, carriers are obtained by crude, uncontrolled crystallisation from various solvents, with significant amounts of material and solvent being wasted. After crystallization, the crystalline carrier is harvested, dried, sieved and comminuted if required. Due to the nature of manufacture the carrier tends to be dense and is of unpredictable shape and surface properties thus leading to batch-to-batch variations in drug deposition. Such inconsistencies can be a burden when working with drugs that must be delivered in precise doses.

It is generally understood that the surface of the carrier particle has areas of roughness (asperities and clefts). The site of an asperity or cleft is believed to be a region of high surface energy. It is these sites, which the drug particles are attracted to, and adhere more strongly to. Consequently, the detachment of drug particles from these sites, upon inhalation, is reduced and uneven, ultimately resulting in unpredictable and reduced deposition of the aerosolised drug particles into the deep lung.

In view of the above problem, it is considered advantageous to reduce the number of these high energy sites available for the drug particle to adhere to. In U.S. Pat. No. 6,153,224, this was achieved by the addition of an anti-adherent material which reduced the adhesion between the carrier and the drug. The respirable fraction of the drug actually achieved using the above composition was as high as 40% with lubricant, glidant or anti-adherent properties, dry mixed with the carrier, have been employed with the aim of reducing the forces of attraction between drug and carrier. Such lubricants are hydrophobic and toxic to the lungs.

Tee et al (1999, Proceedings of Drug delivery to the Lungs X, 33-361) described a process of adding fine particles first, to occupy the high energy sites of the carrier, before the admixture of the drug, this improved drug deposition from 6.3% to 13.4%. Further, WO 95/11666, describes a process rather than physically adding the fine particles, these fine particles were produced in-situ using a milling process, preferably carried out in a ball mill, which alters the surface characteristics of the carrier by removing asperities in the form of small grains that in turn can become attached to the clefts of the surface area of the particles, so saturating the high energy sites. As a result of the preliminary treatment of the carrier, the micronized drug particles are deposited preferentially on lower-energy sites and so are subject to weaker forces of inter-particulate adhesion.

There are, however, disadvantages to such ternary mixes. Including the difficulties of selecting an appropriate process of milling to generate the necessary fine carrier, the type of fine carrier employed, and the sequence and time of mixing. All of which will have an impact on drug content uniformity and increase the complexity of the formulation. In ternary mixes it is extremely difficult to know precisely how much fine particles are needed, and the time during the mixing process when the large carrier particles become saturated with fine carrier. Any excess fine carrier for which there are no available sites on the large carrier particles will cause saturation segregation. Displacement segregation, even in cases when an ordered ternary mix is obtained the differences in particle size of carrier (very fine and coarse carrier) leads to further segregation and the formation of drug rich areas in the mix. (Travers, D. N., Mixing., In Pharmaceutics, The Science of Dosage Form Design; Aulton, M. E., Ed; Churchill Livingstone; London, 1988; 550-563).

U.S. Pat. No. 5,376,386 disclosed a process of producing smooth carrier particles by crystallization from aqueous medium. The resulting particles were found to improve drug deposition.

Further, the shape of the carrier was manipulated to form needles (Larhrib et al, 2000, Proceedings of Drug delivery to the Lungs XI, 18-21). The engineered, elongated carrier particles, despite showing improvements in Salbutamol sulphate deposition, from 5.5% to 22%, the formulations containing these elongated carrier particles produced lower and inconsistent emissions of Salbutamol sulphate from the inhaler device. This was attributed to the poor flow properties of the engineered elongated carrier.

It is generally understood that in carrier/drug compositions, the carrier is present in a much higher concentration than the drug. Thus, despite improvements made in the drug particle design, the overwhelming presence of the carrier will dilute and reduce the effect of the improvements on overall drug delivery. However, by improving the carrier particle design, thus reducing carrier/drug ratios, and promoting the aerosolization of the carrier and the drug particles, drug deposition in the lungs will be increased. Despite this observation, efforts so far to modify the carrier particles have not led to the kind of improvements in deposition obtained by the engineering of drug particles.

At present, conventional commercial carriers are designed to remain in the inhaler device, remain in the mouth or impact at the back of the throat as a result of their high density. However this does not guarantee drug detachment from the carrier nor drug dispersion into primary particles within the airstream. It is the strong adhesion between the drug and dense carrier resulting from the surface roughness and/or presence of crevices on the surface of the carrier, that further impedes drug detachment. In addition the fine carrier added as a ternary component only has a static role, i.e. reducing the adhesion between the drug and the coarse commercial carrier, drug detachment from the coarse carrier is slightly improved but, again, It still does not guarantee drug dispersion into primary particles within the airstream as a result of the cohesiveness of such small drug particles. Thus increasing the amount of the drug particles impacting at the site at which the carrier impacts. Assuming that the problems associated with adhesion and cohesion are alleviated, there are still major problems with the density of the carrier itself. Thus the denser the carrier the more rapid it's impaction caused by it's high inertia. The latter rapidly impacts leaving the drug insufficient time to dissociate from the dense carrier particle. In addition the denser the carrier the greater the inspiratory effort required by the patient to aerosolise the formulation, dissociate the drug from the carrier, disperse the drug into their primary particles in the airstream and entrain the drug particles into deep lung.

In view of the current understanding of the present technology field, there are thus two important issues that the Investigators focused on which can be taken from the teaching of the prior art:
1) The use of High density carrier
2) Lowering the drug-carrier adhesion either by smoothing the surface of the carrier or adding a static ternary component to the formulation.

Apart from particle density and adhesive forces, there are other important factors to consider, which are summarised in a review article (Venables, H. J. & Wells, J. L., Powder Mixing, Drug Dev. & Ind. Pharm, 27(7), 599-612, 2001) highlighted the important factors of carrier and drug particles in powder mixing, drug content uniformity, bioavailability and drug delivery. Some of these features, which include particle density ratios (between the drug particle and the carrier particle) and particle surface texture, are briefly discussed below.

Effect of Particle Shape:

Spherical particles are easier to mix than any other shape. Acicular or flat plate particles prolong the mixing time due to aggregation. Spherical particles have optimal flow properties due to minimal inter-particulate contact and minimises segregation.

Effect of Particle Size:

A homogenous carrier particle size avoids segregation between components in powder formulation. A small particle size and narrow size distribution are required to improve drug bioavailability, however, such small particles give rise to flow problems and segregation caused by the presence of fine particles within the mix. Larger particles act as sieves through which the smaller particles percolate. Matching the particle size of the carrier to drug will improve mixing, content uniformity and reduce segregation. From Zimon's re-suspension model, where it is assumed that the drug slides laterally along the surface of the carrier particle, before it falls off. The longer the drug particle has to travel across the surface of the carrier particle, the greater is the drag force needed to overcome adhesion and friction between drug particle and carrier particle surface (Zimon, A. D., 1982. Adhesion of Dust and Powder, 2^{nd} edn. Consultants Bureau, New York, pp. 307-319). From this, it is understood that small and or spherical particles might be ideal for re-suspension of drug particles.

The size of the particles is a critical factor affecting the site of their deposition, since it determines operating mechanisms and extent of penetration into the lungs. Thus aerosol particles >100 μm generally do not enter the respiratory tract and are trapped in the naso/orpharynx. Particles >10 μm will not penetrate the tracheobronchial tree. Particles must generally be <5 μm in order to reach the alveolar space. On the other hand, particles <0.5 μm in diameter penetrate the lung deeply, but have a high tendency to be exhaled without deposition. However, some studies have found that breath-holding can minimise expiration of small particles (Sandra Suarez and Anthony. J. Hickey. 2000. Drug Properties Affecting Aerosol Behaviour. Respiratory Care, 45 (6) 652-666). Infants, young children, the elderly and patients in acute distress may not be able to hold their breath effectively, even after proper instruction and hence they do not benefit from the pharmacological effect of these fine particles. These fine particles <0.5 μm are pharmacologically advantageous as they penetrate deep into the lungs, tend to be larger in number than larger particles and have high specific surface area that would enable fast and complete drug absorption. Unfortunately these fine particles are generally cleared by exhalation, mucociliary clearance, ingestion and digestion by alveolar microphages. Thus, it is desirable to develop formulations, whereby, such small particles can be maintained in deep lung without discomforting the patient, without being exhaled and without being phagocytosed.

Particle Density:

Various problems can arise when density differences exist between the components of a mix such as increased mixing time coupled with increased propensity for segregation. Gravitational forces pull the denser particles (i.e. carrier) to the bottom, leaving the less dense particles (i.e. drug) on top, in addition vibration will enhance segregation. For the inhalation scenario low density particles are preferred, it is also further preferred that the components of the formulations are of matched densities (i.e. drug and carrier). The latter improves mixing and reduces segregation caused by density differences between the components of the formulation.

Particle Surface Texture:

Porous or rough surfaced particles are suitable for stabilising the mix and ensure uniform drug content uniformity. In the inhalation field porous particles are more suitable than non porous particles of the same size as they have a smaller aerodynamic diameter. The surface nature of the components of the formulation need also to be considered for example their hydrophilic and hydrophobic nature. It has been shown that the deposition of Beclomethasone [a hydrophobic drug] from lactose carrier was dramatically increased when the lactose particles were pre-coated with magnesium stearate [a hydrophobic lubricant] Latent WO 01/05429 A2). In this instance a hydrophobic-hydrophobic surface nature improved the deposition of the drug. The hydrophobic and or hydrophilic nature of the drug and or carrier affects the ease of mixing, stability of the mix and content uniformity.

Ordered Mixing:

The attainment of an ordered mix (i.e. the drug is dispersed on the surface of the carrier) is important in that it prevents segregation of powder mixes. Adhesional forces facilitate the attainment of ordered mixes, in cases where the carrier-drug adhesional force cannot over-come drug-drug cohesive force, or where the carrier particle is extremely small (and milling cannot produce drug particles smaller than the carrier), or where the dose of the drug is very small coating processes are alternatives to producing ordered, uniform and stable mixes.

Drug Concentration:

The drug content variation in a mix increases as the drug content is reduced (as in the case with highly potent drugs), and in such circumstances it is virtually impossible to achieve blend uniformity with a low-dose drug. High particle population is required for low-dose drugs, therefore, particle size control and milling (particle size reduction) are extremely important. However, milling increases cohesion between drug particles, and the agglomerates produced must be deaggregated.

At present, the level of drug deposition possible using a composition comprising a carrier and a drug is limited and can be improved slightly by the engineering of the drug particles, the carrier particles, or both. In view of the limited success achieved so far there is a need for improved engineering of the drug, carrier or both particles.

In order to produce suitable particles there is a need for a method of treating particles in a controlled manner so as to produce particles with the right physical, morphological and/or chemical characteristics, for the drug or carrier with which it is to be associated.

An ideal particle for inhalation or nasal delivery (or in fact any pharmaceutical application) should not have the draw-backs detailed above. An ideal particle should have: suitable adjustable and controlled size range in order to target the particles to the desired region; suitable and controlled aerodynamic diameter (this encompasses particle density, particle size); suitable and controlled particle surface texture; suitable and controlled shape; a narrow size distribution; a crystalline nature; a physical and chemical stability; the capacity for instantaneous and modified release; the ability to allow easy mixing with any other component; the capability of being manufactured on an industrial scale; the capability of comprising up to 100% pure substance; the ability of being free flowing irrespective of the particle size; the capability of being aerosolised on it's own or with a carrier (wherein the aerosolisation is device independent and aerosolised with minimum inhalation effort), a simple and reproducible method of production.

Furthermore the ideal carrier should: be independent of the nature of the drug (i.e. hydrophobic or hydrophilic); not require any further components apart from the drug; stabilise the mix; be versatile (i.e., it is able improve the formulation in which it is included for example improving mixing, improving tabletting properties, improving disintegration time, good diluent, etc . . . ). Adhesion and/or cohesion, which are common problems in dry powder inhalation aerosols, are of less importance or irrelevant in the case of an ideal carrier. The carrier should have good loading capabilities arising partly from it's great specific surface area, thus more drug can be loaded using small amount of the carrier, thus minimising the cost and minimising unwanted side effects. The carrier should help the drug to reach the desired site of action.

The advantage of the present invention is that it gives the user control over one or more of the physical, morphological and/or chemical characteristics needed to obtain a more ideal drug or carrier particle.

### SUMMARY OF THE INVENTION

This invention relates to a new process of creating hairs (projections), pores and controlling the growth of hairs, pores, the particles, changes in physico-chemical properties and their combinations, i.e. architecturing particles.

Accordingly, one aspect of the present invention provides a particle, having at least one changed morphological, chemical or physical feature, wherein said changed feature facilitates the attachment of at least one agent to the outer surface of the particle, thus permitting the particle to act as a carrier for said at least one agent.

Preferably, one of the changed (or engineered) features is either a hair, a pore, a changed hollow volume or an altered particle size. By altering such features various particle characteristics can be controlled, such as altered particle density, altered aerodynamic diameter, altered surface texture, improved flow properties, surface restructuring to reduce cohesiveness.

It will be appreciated that the term hair, within the scope of the present invention, is intended to include any projection from the surface of a particle. Further preferably such hairs may be between 0.001 and 5000 micrometres in length.

Important physico-chemical properties of the hairs, which can be affected to increase the usefulness of the particles, include the type, nature of the agent(s) of which they are composed and the number, surface density, direction of growth and the rate of growth.

There is no restriction on the type of material forming hairs, enabling instantaneous, controlled or sustained release profiles, in order to suit the intended use. The hairs can be produced from a suitable and safe (generally recognised as safe “GRAS”) penetrating enhancer agent to achieve the intended pharmacological effect of the therapeutic agent.

The nature, quantity, the length and the physico-chemical properties of the hairs can be engineered in a controlled fashion to suit the intended use, to give for example increasing specific surface area, thus more therapeutic agent can be loaded onto the hairs reducing the quantity of carrier required compared to the conventional carrier used in the current dry powder inhalation aerosol.

The present invention is contrary to the general trend of the prior art, where rough surfaces have been seen as a burden for traditional dry powder inhalation, such rough surfaces in this invention are advantageous (see below). In fact the present invention seeks to promote roughness of the particle surface (or asperities) by the presence of projections (hairs) and/or pores.

The presence of hairs maintain the stability and content uniformity of the mix. The hairs also minimise the contact between the carrier particle core and the therapeutic agent. The hairs are part of the carrier, however, they act as a ternary component minimising full contact between the carrier core and the therapeutic agent. This is a new concept contrary to the prior art, where a ternary component (such as fine carrier) is added as a static component only to reduce the adhesion between the therapeutic agent and carrier. The density of the hairs can be adjusted to enable the hairs to oscillate or vibrate (in this instance hairs acts as a dynamic ternary component, which is contrary to the prior art when the standard ternary components is static) when the particles are subjected to inhalation. Low adhesion of therapeutic agent particles to the hairs combined with efficient oscillation of the hairs allow better detachment of the therapeutic agent particles, the remaining therapeutic agent particles attached to the hairs and/or to the carrier core will travel with the low density carrier. The increased surface area conferred to the particle by hairs automatically improve aerodynamic properties of the particle, in that the inhalation air flow acts on this surface area to propel and suspend the particle in the airstream. Hence, the greater the surface area of the particle the greater the propulsion and suspension of the particle.

Furthermore, the hairs can be produced from a bioadhesive agent which at the point of carrier impaction allow the hairs to act as grappling hooks anchoring the carrier-therapeutic agent particles or therapeutic agent particles to the impact site of the lung epithelia allowing the therapeutic agent to be released and absorbed. This concept is important for delivering very small particles below 0.5 micrometers such as liposomes to the lung, this particle size range is known to be cleared from the lung, whereas the presence of hairs will maintain them at the impact site until they have released their therapeutic agent pay load. Hence the presence of hairs will maintain particles lower than 0.5 micrometers at the impaction site, preventing their expiration, muco-cilliary clearance or ingestion and digestion by macrophages. Such small particles are pharmacologically advantageous as their large surface area to volume ratio give superior and faster absorption. The hairs increase the residence time of the particles by maintaining the particles at the impact site. Thus the therapeutic agent concentration at the site of action is higher and this is appropriate for a locally acting therapeutic agents where biological activity is dependent on therapeutic concentration at the site of action. These small size ranges are thus made pharmacologically useful with this technology whilst in traditional formulations these size ranges are usually cleared from the lungs and are consequently of no therapeutic or economic value due to their wastage.

Preferably, the particle of the present invention will have a low density and have hairs on the surface of the particles.

Preferably the density of the particle may be reduced by increasing the hollow volume of the particles.

Advantageously, the particles may be spherical in shape. This regular shape coupled with low density of the carrier allow better technical handling and easier and total aerosolisation of the powder. When the particles are used in dry powder inhalation, the regular spherical shaped particles flow better allowing consistent filling and better emptying during inhalation.

The preferred particle size is between 0.05 μm and 4000 μm in diameter.

According to the present invention the lower density of the carrier particle, the spherical shape of the particle together with the presence of hairs on the particle surface, improved the aerodynamic properties of the particles, facilitating their easy aerosolization from a dry powder inhaler device (and the emitted particles will travel further in the air stream despite changes in air stream velocity).

The low density of the carrier facilitates a long flight time which in turn allows more therapeutic agent particles to detach, oscillation of the hairs promotes further detachment of therapeutic agent particles from the carrier whilst those therapeutic agents particles which do not detach from the carrier particles are carried to deep lung to the impact site of the light, low density carrier particle. The bioadhesive and anchoring functions of the hairs retain the therapeutic agent at the lung epithelia for sufficient time to enable therapeutic agent transfer to the lungs. The carrier particle of the current invention delivers more therapeutic agent to the site of action compared with the traditional high density carrier, that usually remains in the inhaler device or impacts in the mouth.

Advantageously, the engineered carrier may be composed of 100% therapeutic agent, thus allowing the therapeutic agent to be delivered on its own or act as a carrier for one or more therapeutic agent particles The carried therapeutic agent particles can be traditionally prepared or engineered according to the current invention.

When a potent therapeutic agent is used in a mix, the amount of therapeutic agent used is small and to ensure a uniform mixture it is necessary to increase the number of therapeutic agent particles per sample or dose. To do this it is necessary to use a smaller therapeutic agent particle size, however, producing such very fine powder is difficult and often attended by severe aggregation (using conventional milling, spray drying or crystallisation techniques) thus defeating the object of size reduction in the mixing process. When the proportion of therapeutic agent is extremely small and finally presented in a small dose unit, physical dry mixing of solids will fail to produce an adequate dispersion of therapeutic agent within the formulation. To avoid the above drawbacks, the current invention adopts an efficient and reproducible strategy in which the therapeutic agent is maintained in a liquid. The resulting therapeutic agent-liquid mix is reduced in size by atomisation to form a fine mist. This fine mist contains individual liquid droplets whose size is much smaller than that obtainable by conventional milling such as micronisation and spray-drying. Furthermore these liquid droplets are uniform in size and therapeutic agent content. Using the right atomisation protocols, the size of the liquid droplets can be arranged to be several orders of magnitude smaller than that of carrier particle with which it is mixed. Mixing of the liquid droplets and carrier results in an efficient, uniform and stable mix. The therapeutic agent adhered to the particle is uniformly distributed and smaller in size than the carrier. The therapeutic agent particles can consequently travel with the low density engineered carrier to the deep lung. The small size of the therapeutic agent particle enables fast dissolution and transport in lung epithelia. Hence, the problems of adhesion and cohesion encountered in traditional dry powder inhalers is of no consequence with this invention which is in direct contradiction to the current state of the art.

Advantageously, one or more agents (one or more of which may be therapeutic), carried in a liquid or vapour-loaded state can be transferred to the particle. This liquid state, vapour-loaded state and transferred agent corrects surface defects, restructures the surface of the particles which in turn reduces the cohesiveness of the particles, alters the particle density, particle size and thus their flow properties. The transferred agent is uniformly distributed to the particles forming a stable and homogeneous mix. The preferred vapour-loaded states for small quantities of agent transfer include mist, droplets, foam, spray, steam, fog or vapour. The vapour-loaded transferred state is more efficient and effective than conventional methods of mixing dry micronised powders.

Further the particles adhered to the carrier can be present on the carrier as discrete, discontinuous or continuous particles or films. Apart from transferring agent particles to the carrier, the method of transfer using the current invention can be manipulated to also change at least one or combinations of one or more morphological, chemical or physical features of the particle and/or, transferred agent according the above. In addition the change of at least one or combinations of one or more morphological, chemical or physical features of the particle can be manipulated to occur before, during or after the transfer of the agent.

According to the present invention an agent can be selected that imparts to the resulting particles a plastic nature, as it is known that materials that are plastic in nature deform plastically (with the possibility of shape change) rather than fracturing or elastically deforming. The majority of therapeutic agents used in pharmaceuticals tend to have elastic or brittle (i.e. fragmenting) behaviour. Particles intended for deep lung administration may be preferred to have a plastically deforming component as such these particles absorb and transfer the energy of impact to plastic deformation preventing bouncing of the particles from the site of impact in the lungs. This plastic deformation may also lead to a change in shape of the particle further increasing the contact area between the particle and the lung at the site of impaction thus increasing the drug absorption. This phenomena is extremely important for fine powder (i.e. particles less than 1 micrometer), even though they penetrate deeply into the lung, they are exhaled partly because these particles have bounced off the surface of the lung epithelia. Thus giving the particles a plastic behaviour will prevent bouncing of the particle, instead keeping them at the impact site thereby maximising therapeutic and minimising wastage and unwanted side-effects. Furthermore, the plastic nature of these particles are more stable during processing, packaging and shipping as they are mechanically tough and are less likely to abrade than material that are brittle in nature. Examples of agents that impart a plastic nature to the particles are Polyvinyl alcohol, Polyvinylpyrrolidone and polyethylene glycol (PEG).

According to the present invention the selected agent may impart to the particle a brittle nature such that when these particles impact in deep lung the particle fragment into smaller particulates, these smaller particulates are then spread over a larger area compared to the initial impact site thus increasing drug absorption. In the present invention the brittle behaviour of lactose was enhanced by forming very fine and weak projections. These projections upon impact fragment giving ultra-fine powder that increases the surface area of contact between these particles and the lung.

Another aspect of the present invention provides a method of producing engineered articles for use alone or as carriers for one or more agents, comprising the steps of:
a) processing at least one agent to form a particle;
b) treating by making available a fluid alone or in combination with at least one additive to the particle to promote changes in one or more of the morphological, chemical or physical features of the particle;
c) repeating steps (a) to (b) as many times as necessary;
d) harvesting engineered particles;
e) repeating steps (a) to (d) as many times as necessary.

The present invention provide specific examples, numbered 1 to 18, of engineering treatments which produce engineered particles for use as agent carriers substantially as described herein with reference to the examples.

The severity of the treatment conditions and the time of treatment determines the extent and the degree to which there are changes in the particles morphological, chemical or physical features. The severity of the treatment conditions are controlled at least by the time of treatment, state of matter in which the particle meets the state of matter of the fluid and the addition of an agent or additive.

Preferably, the promoted change of step (b) results in at least one change to the particle from a list consisting of but not limited to: promoting the growth of hairs; modifying the properties of the existing hairs; promoting the formation of pores; modifying the properties of existing pores; and increasing the hollow volume of the particle.

Preferably the fluid used in the above method contains at least one medium that promotes changes in any of the morphological, chemical or physical features of the particle and/or aiding the transfer of an agent to the particle. Further preferably the fluid is either aqueous, organic, or a combination thereof. It is also preferable that the fluid comprises either water, acetone, ethanol, or combinations thereof.

Preferably during the step of treating the particle with fluid, the fluid is introduced to the particle either in bulk, as droplets, as a foam, as a mist, as a vapour, as steam or combination thereof. The particles and fluid can be static or in motion or combinations thereof.

Alternatively, during the step of treating the particle with fluid, the particle is introduced to the fluid either in bulk, as droplets, as a mist spray, as a vapour or as a steam or combinations thereof. The particles and fluid can be static or in motion or combinations thereof.

It is appreciated that it may be advantageous if at least one further agent is added to the fluid before and/or during and/or after the particle has been treated with the fluid.

Suitable states of matter for the particle and fluid include: solid, frozen, liquid, gas (ideal, real or mixtures thereof), vapour, supercritical fluids, solutions, suspensions, dispersions, emulsions or micro-emulsion, colloids, liquid crystals, visco-elastic, gels, slurry, paste, semi-solid, molten or combinations thereof.

In the above method additives are introduced to facilitate the particle engineering process. Such additives can be environmental or non-environmental. Preferable environmental additives include: heat, moisture, vacuum, radiation, pressure, shear forces, magnetic forces, vibration, stirring, vortexing, mixing, tumbling, centrifuging, masticating, ultra-sound waves or extruding, electrical, or combinations thereof.

Further preferably, at least one selected additive in the above invention is stirring. Another preferable additive in the above method is the maintenance of heat in the range −200 to 200° C.

Agents can be used to facilitate the particle engineering process, such agents are preferably polymers. Preferably such polymers are biodegradable or erodible. Further preferably, suitable polymers are selected from a group consisting of polyvinyl alcohol, polyvinylpyrolidone and polyethylene glycols.

In the above method, the step of treating may preferably last for between 1 microsecond and 30 minutes.

The method of the present invention maintains the particle shape whilst adjusting the particle size distribution to the desired particle size range. This can be easily achieved without changing the operating parameters used to produce the untreated original particles.

It is appreciated that the particles of the present invention may be used to deliver therapeutic agents via a range of routes, such routes preferably include: pulmonary, oral, parental, nasal, rectal, tonsillar, buccal, intra-ocular, topical/transdermal, or vaginal.

The respiratory anatomy has evolved in such a way as to actively prevent inhalation of airborne particulates. The upper airways (nose, mouth, larynx and pharynx) and the branching anatomy of the tracheobronchial tree acts as a series of filters for inhaled particles. Thus particles >100 micrometer generally do not enter the respiratory tract and are trapped in the naso/oropharynx. Particles greater then 10 micrometer will not penetrate the trancheobronchial tree. Particles must generally be <5 micrometers in order to reach the alveolar space “(Suarez S. and Hickey. A. J 2000. Drug Properties Affecting Aerosol Behaviour. Respiratory Care, 45 (6) 652-666). This current invention enables control of particle size, particle density and particle surface texture in order to control the aerodynamic properties of the particle, and from the above, such resultant particle(s) can be used to specifically target a particular region of the respiratory tract. With the current invention, particles targeting multiple sites of the respiratory tract may be formulated together to enable all the sites to be targeted at once or formulated individually to target only one region of the respiratory tract. The particles designed for a targeted region can be used as carrier for particles designed to target a region different from the carrier.

The method of the present invention enables control of the changes in morphological, chemical or physical features whilst transferring an agent to the particles at the same time.

The particles of the present invention are advantageously used to deliver any of the agents selected from a group comprising: therapeutic agents, prophylactic agents, diagnostic agents, excipients, diluents, flavourants, fragrances, dyes, nutrients, sweeteners, polymeric drugs, proteins, lipids, organic substances, inorganic substances, pro-drugs, antigens, and combinations thereof.

Preferable therapeutically active agents include: corticosteroids, antiinflammatories, antitussives, bronchodilators, diuretics, anticholinergics, hormones, analgesics, vaginal preparations, antiallergics, anti-infectives, antihistamines, anti-neoplastic agents, anti-tuberculous agents, therapeutic proteins, and peptides and derivatives thereof.

A further embodiment of the present invention provided a low density carrier particle having hairs on the surface thereof, wherein the particle acts as a carrier for the delivery of agents that are either anti-inflammatory drugs, bronchodilator drugs or a combinations thereof into the lungs of a patient via dry powder inhalation.

It is appreciated that in some circumstances it may be advantageous for the particle itself to be a therapeutic agent. Therefore, the principles applied to produce the carrier particle can also be applied to produce particles of the therapeutic agent so that these therapeutic agent particles, can advantageously, be delivered on its own or combined with one or more carriers. The latter carrier can be conventionally prepared carrier or carriers prepared according to the present invention or conventionally prepared therapeutic agent or therapeutic agents prepared according to the present invention.

Fluticasone propionate, beclomethasone dipropionate and salbutamol sulphate and combinations thereof are a selection of therapeutic agents that can be advantageously delivered using the hairy, low density and porous, particles of the present invention.

Preferably, where the method includes the set of adding a further agent to the fluid/particle mix during the treatment of the particles such agent is beclomethasone, fluticasone or salbutamol sulphate.

Of the above agents, combinations of beclomethasone and lactose, fluticasone and lactose, polyvinyl alcohol and lactose, or polyvinylpyrolidone and lactose, or even lactose on its own, are considered most preferable.

The morphological features of the architectured particles are assets that enable, for example, the entrapment and increasing the shelf-life of perfumes, flavourings, and taste-masking agents.

Hairs can be surfactants retarding drug creaming from suspension, enhance solubility in certain media, lubricate the valve and it's components during the depression and release cycle associated with container emptying of Metered Dose Inhalers, stabilising the suspension, minimising friction between particles, minimising drug particle adhesion to container walls, dispersing particles within the medium, prevent caking and maintain a homogenous drug particle size.

This technology has great applicability in other areas, for brevity, such examples are nasal delivery, tablet formation technology, delivery of controlled systems such as liposomes. This invention, from a knowledge of the route and site of delivery for the agent, knowing the requirements for that route of delivery and the physico-chemical properties of the agent to be delivered, thus, this invention brings solutions for designing suitable particles required for that route and site of delivery.

Patent WO 00/27373 highlighted the important properties of particles for nasal delivery. In the latter patent, 85% of the therapeutic agents particles to be delivered have a size over 5 μm and at least 90% have a size less than 20 μm, and when mixed with an excipient 90% of the particles are of a size less than 10 μm. The particles of the present invention fulfil this criteria and are thus applicable for nasal delivery. Furthermore, the particles of the present invention can be engineered to include a mucoadhesive penetrating enhancer component that maintain the particles at the site of action without being cleared from the nose and promote absorption and transportation through the nasal mucosa. Particles for nasal mucosa should not be smooth as such particles may bounce off the nasal mucosa, whilst particles with projections, such as described in this patent will anchor onto the nasal mucosa and by virtue of the mucoadhesive incorporated within the particles and this will keep the particles in contact with the nasal mucosa. Particles with projections will be retained also by the nasal hairs further increasing the deposition of therapeutic agent to the nasal mucosa.

In tabletting technology it is well known that optimal tablets are obtained when plastically deforming and fragmenting materials are compacted together. The plastically deforming and fragmenting material are usually prepared as a physical mixture, to this physical mixture the drug is added before tabletting. However, in this instance a uniform formulation cannot be assured and de-mixing and segregation always occurs. The particles of the present invention can be successfully used in tabletting as the plastically deforming PVP, fragmenting lactose and drug are incorporated into one particle, assuring formulation uniformity hence better compressibility than physical mixtures, minimising the processing time, minimising the cost compared to labour and cost intensive wet and dry granulation techniques routinely used in tablet technology. Furthermore, the particles of the present invention are spherical, hence they flow better from the hopper into the die and pack easily. In addition, their hollow nature (less resistive to the compression force) enabling the particle to collapse under low pressure so that the fragmenting and plastic-deforming components come into play at an earlier stage of the compression cycle. Since low compression forces are commonly employed for the particles of this invention, there is increased longevity of the tabletting machine and tooling. Furthermore, the resulting tablets are less friable and disintegrate faster which are both desirable properties in tablet manufacture.

The particles of the current invention can be advantageously formulated in a suspension, this suspension can be used for example in oral dosage forms, topical dosage forms, parenteral dosage forms and the like. The particle as shown in the examples and detailed description, are below 5 micrometers to ensure a slow rate of sedimentation of the suspended particles. The engineering process allows the density of the particle to be matched with that of the dispersing medium. The particles are isometrical in shape and of narrow size distribution allowing the particles to settle at similar velocities in the dispersing medium to prevent phase separation of the particles and the dispersing medium. Further the engineered pores of the particles allow the flow of the dispersing medium into and around the particles. The flow of dispersing medium into the hollow volume of the particles not only minimises the density differences between the particle and the dispersing medium but it help support, suspend and maintain the particle in the dispersing medium thereby sedimentation of the particles within the dispersing medium is reduced.

Liposomes or nano-particles are usually made from waxy materials such as surfactants and are consequently delivered by wet nebulisation but never by dry powder aerosolisation using conventional technologies. This is due to the high liposome-liposome (or nano-particle-nano-particle) cohesion and adhesion forces. However, these high adhesive forces are unimportant with the method of engineering particles of the present invention (i.e. light hairy carrier particle and liposome or light hairy carrier and nanoparticle) and deep delivery of drugs into the lungs is made possible. Liposomes are ideal carrier systems in that they are hydrophobic, which will be quickly and easily absorbed and transported by the hydrophobic lung epithelia. Liposomes, generally, are usually opsonised or phagocytised hence they have a short biological half-life. Application of this process to produce hairs on the surface of liposomes increases their biological half lives and hence improves their pharmacological usefulness.

Previously, it was stated that the carrier could be designed with morphological features which actively promote drug detachment from the carrier. One such morphological feature has been commented in U.S. Pat. No. 5,869,098, however, the author's failed to realise its importance. It was used in this patent purely as a reference to indicate the cessation of crystallization. However this invention is specific in actively seeking to produce this feature as one of the important part of the engineered particles. This feature as described, U.S. Pat. No. 5,869,098 is “fine cat whisker-like needles and tiny blades which grow and project along the surface”.

Although the present invention, which comprises a multitude of techniques, shares one technique, that is immersion of solids in solvents, with U.S. Pat. No. 5,869,098, the aim and resultant product is completely different and possess several advantages compared to U.S. Pat. No. 5,869,098.

The principle events in U.S. Pat. No. 5,869,098 are:
1 production of floss using high temperature and high shear forces;
2 Chopping of the floss;
3 Addition of additive, which may be bioactive that may act as a nucleating agent;
4 Immersion or addition of organic solvent (with or without the presence of water), or subjection of the floss to organic solvent vapour, with the intention of producing a crystalline material;
5 The final recovered product is in the form of spheroidal micro-crystallites that essentially consist of agglomerated rods in the form of a “dome or raspberry like structure”;
6 The product is aimed as a fondant comestible;
7 Suggest the use of the resultant particles as inhalants.

For the present invention, addressing items 1-7 above. A floss is not produced, in fact the starting material need not even be processed but can be used in the raw state. Contrary to U.S. Pat. No. 5,869,098, the staring material for this invention is not limited to those that are amorphous, crystalline materials can also be processed with the same results. There is also no limitation on the starting shape or size. The floss is amorphous in nature and consequently is thermodynamically unstable hence it was necessary to process it at comparatively low temperatures otherwise it's structural integrity was destroyed.

Processing the floss at elevated temperature will ultimately lead to uncontrollable crystallization and floss destruction. Whereas, in this present invention the application of heat is desirable, in that it reduced the processing time, it facilitated pore and hair forming processes whilst increasing internal hollow volume and pore size. In addition the solid nature of the starting material is more resistant to elevated temperatures compared to the floss and the elevated temperature enabled the starting material to grow in an isometric manner, whilst maintaining it's high degree of mono-dispersity and maintaining the shape of the original particle.

Since the floss is made from sugars, this limits the starting materials to sugars. The spheriodal micro-crystallites of U.S. Pat. No. 5,869,098 are not true spheres but agglomerates of rod like crystals. Whereas, in the present invention the particles retain the original shape of the starting material. Since the final product of U.S. Pat. No. 5,869,098 is much denser than the starting floss these particles may be undesirable for inhalation as light particles are required (as discussed above). In addition for inhalation, these micro-crystallites must disperse in the inhaler device and given the fact that this dispersion occurs in saturated sugar solutions, this would be difficult to obtain in an inhaler device in the dry state. Furthermore the rough surface characteristics of the micro-crystallites would impede drug detachment. All of these limit the use of such particles in dry powder inhaler devices. The present invention produces aerodynamically favourable particles with low bulk density that can be delivered as a whole to deep lung rather or as fragments. In addition the present invention can deliver the drug alone without the need of a carrier, whilst U.S. Pat. No. 5,869,098 needs re-crystallised floss as carrier.

U.S. Pat. No. 5,869,098 whilst re-crystallising amorphous material increases it's bulk density and lowers the corresponding specific surface area. In contrast, the present invention increases the specific surface area and decreases the bulk density. Also in the present invention the number, size the density and other characteristics of the hairs can be manipulated to achieve the requirements for that application.

### BRIEF DESCRIPTION OF THE DRAWINGS

In the drawings, which show various preferred embodiments of the invention:

FIG. 1 shows a scanning electron micrograph of Spray-dried lactose particles (before treatment with ethanol);

FIG. 2 shows a scanning electron micrograph of lactose particles of FIG. 1 after exposure to hot ethanol (45° C.) for 10 min;

FIG. 2.1 shows a photograph of a fruiting head of dandelion fluff;

FIG. 3 shows a scanning electron micrograph of an aggregate of hairy lactose particles after exposure to hot ethanol for 10 min;

FIG. 4 shows a scanning electron micrograph of lactose particles of FIG. 1 after exposure to 50 ml ethanol vapour;

FIG. 5 shows a scanning electron micrograph of pre-treated lactose particles of FIG. 4 after treatment with boiling ethanol for 10 seconds;

FIG. 6 shows a scanning electron micrograph of spray-dried lactose particles after treating with boiling ethanol for 60 seconds;

FIG. 7 shows a scanning electron micrograph of spray-dried lactose immersed in ethanol at ambient temperature for 45 minutes. Note that these particles have micron-size hairs;

FIG. 8a shows an original spray-dried lactose particle before treating with ethanol vapour;

FIG. 8b shows spray-dried lactose particles after treating with 50 ml ethanol vapour;

FIG. 8c shows a golf-ball type surface textured lactose particle with nano-projections were obtained after treating with 120 ml ethanol vapour;

FIG. 9 shows a scanning electron micrograph after treating vapour treated partially architectured lactose particles, shown in FIG. 8c, with hot ethanol for 40 seconds;

FIG. 10 shows spray-dried lactose-PVA particles before treating with boiling ethanol;

FIG. 11 shows a scanning electron micrograph of lactose-PVA particles after treating in boiling ethanol for 30 seconds;

FIG. 11.1 shows a scanning electron micrograph of lactose-PVA particles after treating in hot ethanol for 60 seconds;

FIG. 12 shows a scanning electron micrograph of spray-dried lactose-PVP particles;

FIG. 13 shows a scanning electron microscopy of spray-dried lactose-PVP particles after treating with boiling ethanol for 60 seconds;

FIG. 14: Frozen Lactose particles obtained by freezing lactose droplets in liquid nitrogen (the scale bar is that of a 15 cm ruler);

FIG. 14.1: Lactose particles obtained by freezing lactose droplets followed by treating, for 5 mins, with ethanol/acetone mixture containing PVP as an excipient;

FIG. 15 shows a scanning electron micrograph of lactose particles obtained by freezing lactose droplets followed by treating these droplets, for 5 minutes, with ethanal/acetone mixture containing PVP as an excipient;

FIG. 15.1 shows the detailed structure of a particle shown in FIG. 15;

FIG. 16 shows a scanning electron micrograph of lactose particles coated with fluticasone hairs;

FIG. 17 shows a scanning electron micrograph of lactose particles, which have been vapour-architectured twice with ethanol alone;

FIG. 18 shows a scanning electron micrograph of lactose particles which have been vapour-architectured twice with ethanol alone, followed by a third vapour architecture with ethanol/water mix;

FIG. 19 shows a general view of spray-dried lactose-PVP (24,000 MW) particles;

FIG. 20: Scanning electron micrograph of lactose—PVP particle architectured with ethanol vapour containing BDP (as an agent);

FIG. 21 shows a scanning electron micrograph of spray-dried lactose particles architectured by a fine mist formed from a 94/6% ratio of ethanol/water containing lactose;

FIG. 22.1 Particle size distribution of untreated Microfine lactose using a Sympatec Helos Particle Size analyser at 1 Bar dispersion pressure;

FIG. 22.2 Particle size distribution of treated Microfine lactose (treated with Liquid Nitrogen vapour) using a Sympatec Helos Particle Size analyser at 1 Bar dispersion pressure;

FIG. 22.3 Particle size distribution of treated Microfine lactose (treated by immersion in Liquid Nitrogen) using a Sympatec Helos Particle Size analyser at 1 Bar dispersion pressure;

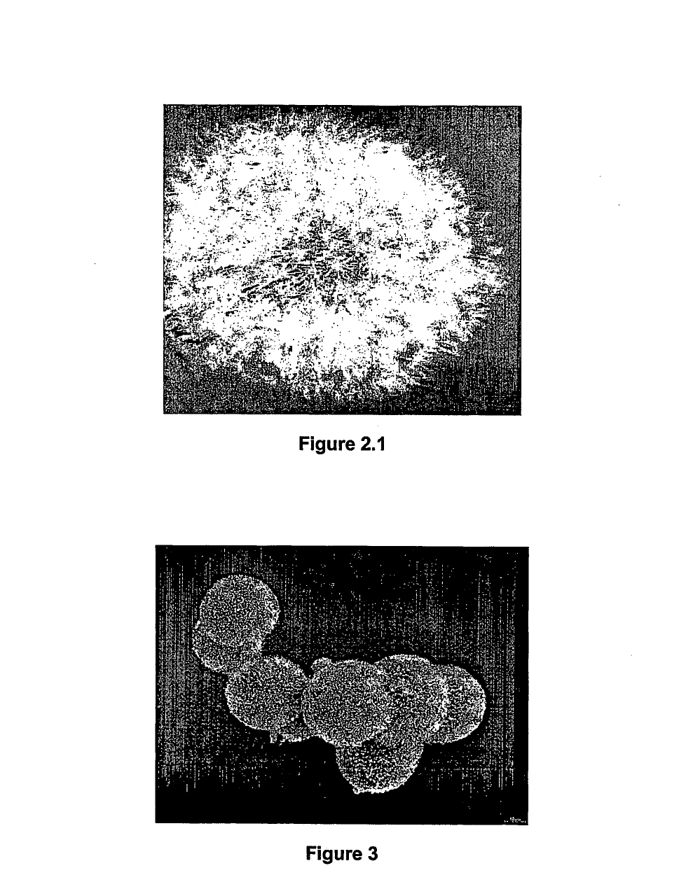

FIG. 2.4 Particle size distribution of treated Microfine lactose (treated by a combination of immersion in Liquid Nitrogen and treatment with Liquid Nitrogen vapour) using a Sympatec Helos Particle Size analyser at 1 Bar dispersion pressure;

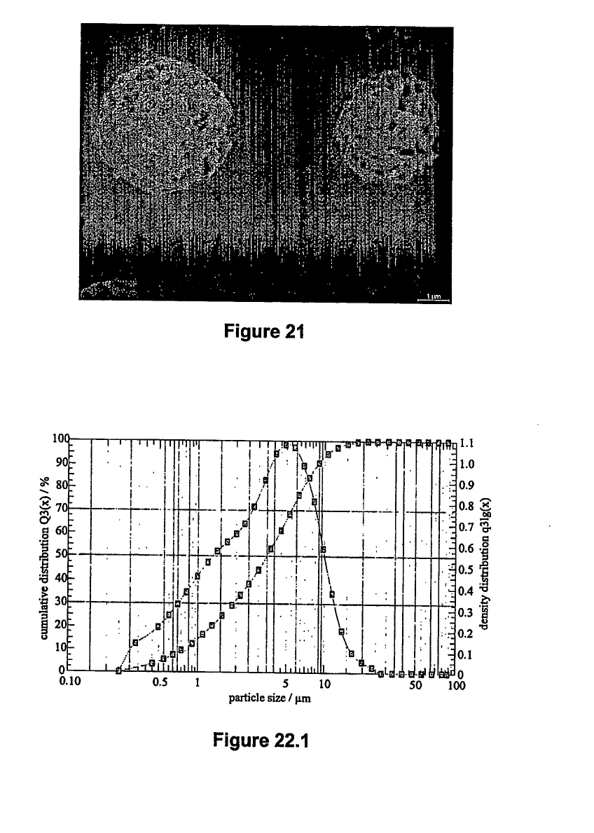

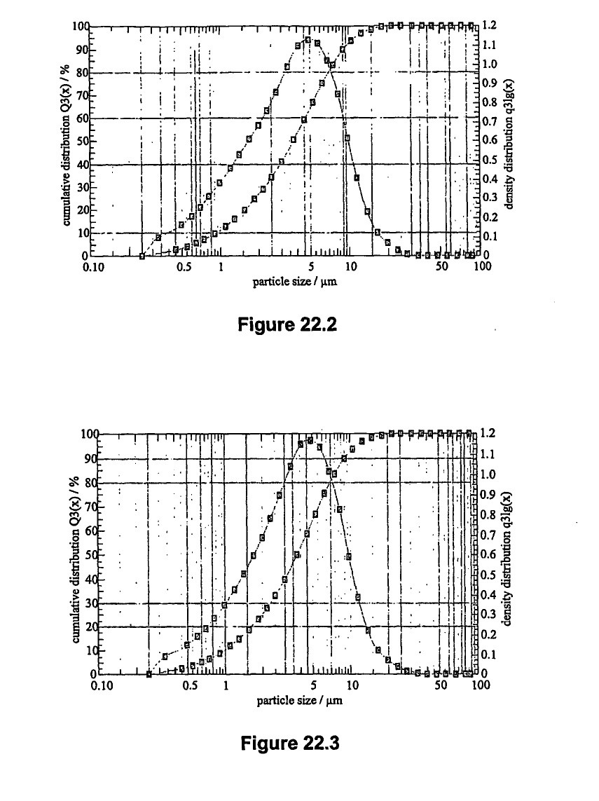

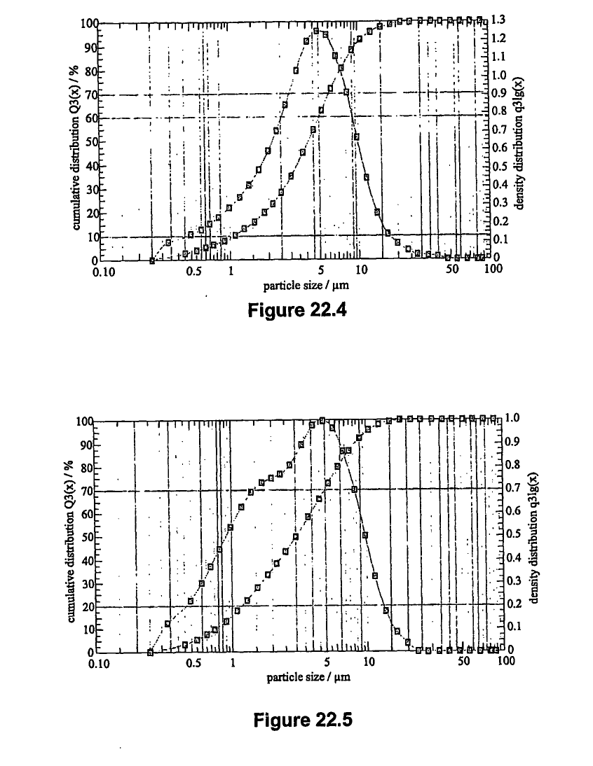

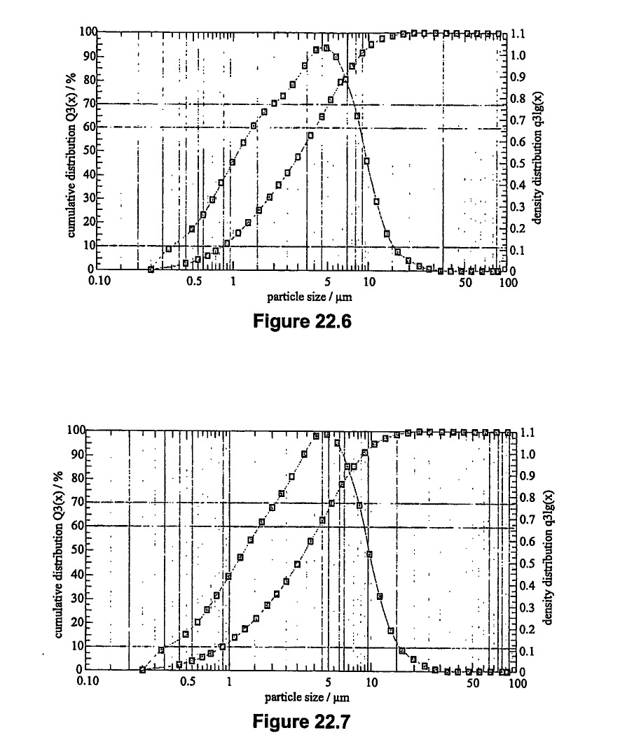

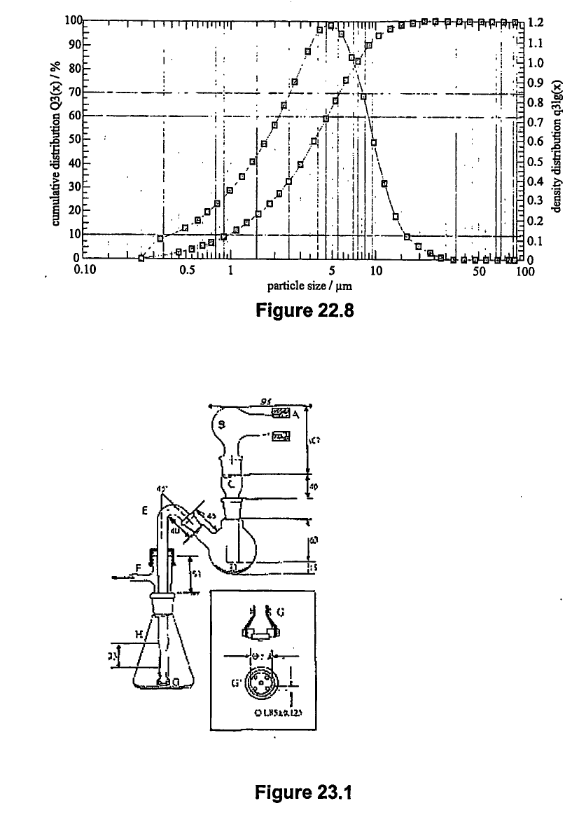

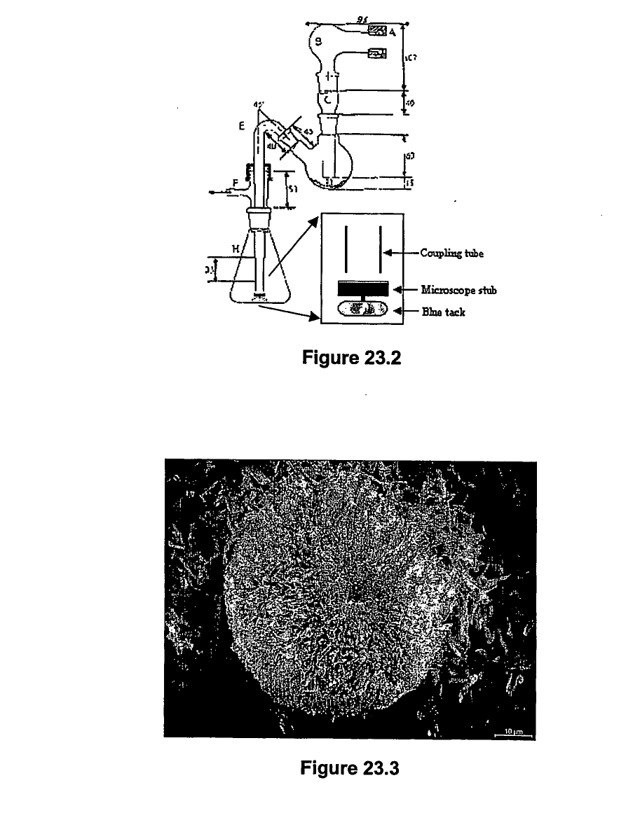

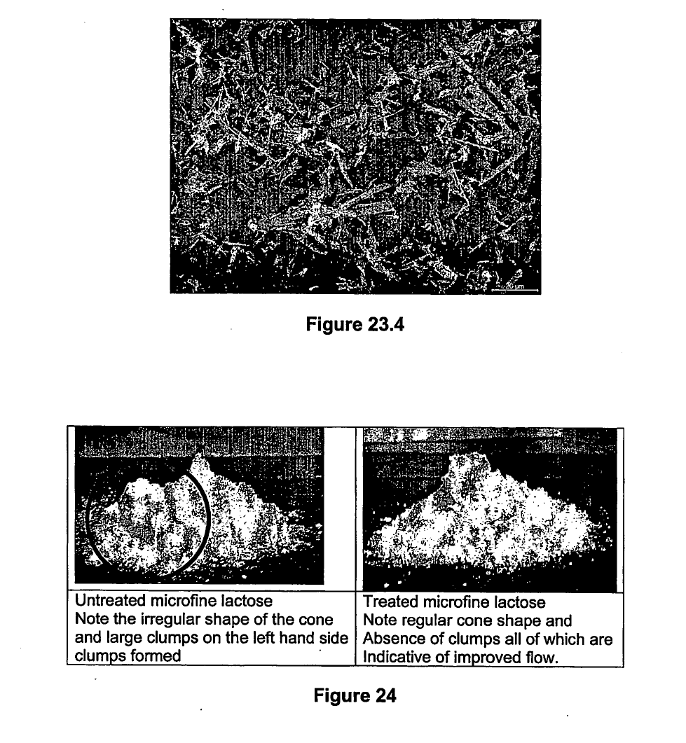

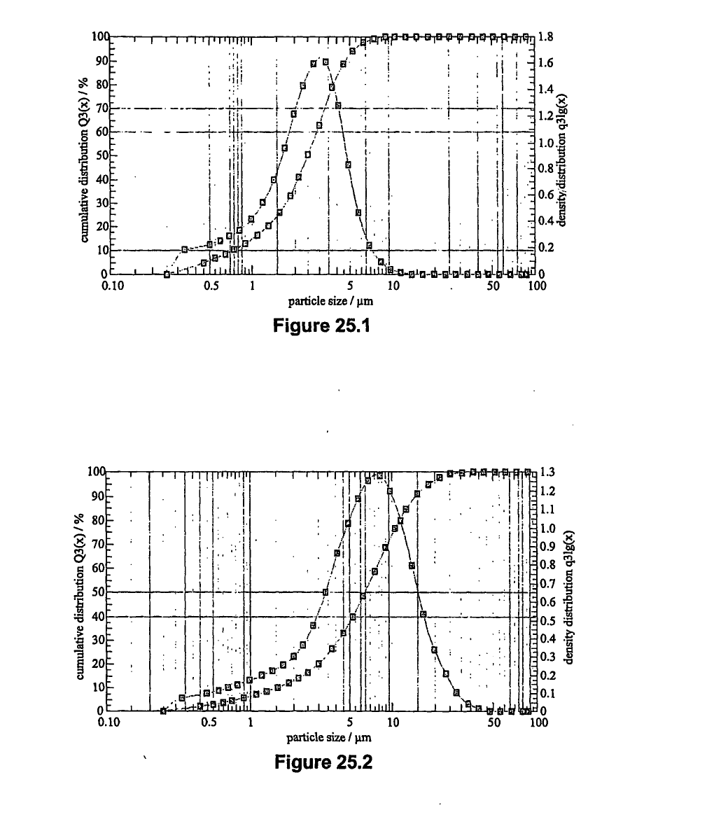

FIG. 22.5 Particle size distribution of untreated Microfine lactose using a Sympatec Helos Particle Size analyser at 3 Bar dispersion pressure;

FIG. 22.6 Particle size distribution of treated Microfine lactose (treated with Liquid Nitrogen vapour) using a Sympatec Helos Particle Size analyser at 3 Bar dispersion pressure;

FIG. 22.7 Particle size distribution of treated Microfine lactose (treated by immersion in Liquid Nitrogen) using a Sympatec Helos Particle Size analyser at 3 Bar dispersion pressure;

FIG. 22.8 Particle size distribution of treated Microfine lactose (treated by a combination of immersion in Liquid Nitrogen and treatment with Liquid Nitrogen vapour) using a Sympatec Helos Particle Size analyser at 3 Bar dispersion pressure;

FIG. 23.1:Standard twin stage impinger (TSI) of Apparatus A as described in the British Pharmacopoeia, BP 2001;

FIG. 23.2: mTSI showing the attachment of the coupling tube to the microscope stub;

FIG. 23.3 Scanning electron micrograph of engineered long time of flight, hairy lactose particles, having an aerodynamic diameter less than 6.4 micrometers, deposited on the lower stage of a modified twin stage impinger. Note that the geometric diameter of the particle is at least 60 micrometers;

FIG. 23.4 Scanning electron micrograph of lactose hairs and fragmented hairy lactose particles having an aerodynamic diameter less than 6.4 micrometers;

FIG. 24 Comparison of cones formed by untreated microfine lactose and microfine lactose treated according to the fourth embodiment;

FIG. 25.1 Particle size distribution of untreated Spray-dried lactose using a Sympatec Helos Particle Size analyser at 1 Bar dispersion pressure; and

FIG. 25.2 Particle size distribution of Spray dried lactose vapour architectured using 260 ml of ethanol with a Sympatec Helos Particle Size analyser at 1 Bar dispersion pressure.

### DETAILED DESCRIPTION OF THE INVENTION

The present invention exists in more than one embodiment. The first embodiment of the present invention is provided in the form of a method of treating particles to enhance their ability to perform certain functions, but particularly the delivery of drugs to a target region in a patient. The method of the present invention enables particles to be engineered with the appropriate chemical, morphological and/or physical features for any particular task.

A second embodiment of the present invention exists in the form of the particular particles engineered by the controlled treatments of the above mentioned method. This second embodiment details typical particle and powder engineered features that are controllable using the methods of this invention. It is appreciated that such particles can be engineered to deliver an active agent, e.g. a therapeutic agent, to a target region of a patient. The types of engineered features is dependent on the type of agent being transported and the chosen pathway of the delivery. Typical delivery routes are considered to be oral, parenteral, nasal, pulmonary, rectal, tonsillar, buccal, intraocular, topical/transdermal, vaginal. However, the preferred administration routes are oral, nasal, pulmonary and rectal.

A third embodiment of the present invention relates to a particular family of therapeutic agent delivery particles, i.e. carrier particles for the delivery of drug via inhalation in to the lungs. The carrier particles of this embodiment have specific engineered features which make them better suited for the delivery of drugs deep into the lungs. The present invention provides carrier particles with improved lung deposition of therapeutic agents. These improved carriers are low in density and tend to have non-smooth (e.g. containing pores or hairs according to the invention) surfaces. As was discussed earlier in this document, it is appreciated that both of these characteristics run contrary to the accepted prior art.

A fourth embodiment of the present invention is the application of the method of the invention for micronising, mixing and achitecturing particle in one step. This embodiment relates to a process of micronising without using conventional milling, spray drying or conventional crystallisation techniques to produce ultra fine particles. These ultra-fine particles are attached to particles of the same size or of larger size to form a stable uniform mix avoiding segregation. Another aspect of fourth embodiment is the introduction of the particles into bulk fluid

A fifth embodiment of the present invention is the application of the method of the invention in an innovative MELT BACK crystallisation process to produce particles with the required physico-chemical and morphological features.

In order for the present invention to be understood the following guidelines for carrying out the method of the invention are given below, followed by examples of more specific methods of treating particles to engineer desirable chemical, morphological and/or physical features.

The method of the present invention provides a means of engineering particles to give them particular morphological, chemical and/or physical features and these such features imparted to particle improve their formulation and delivery capabilities.

Important formulation capabilities include surface restructuring to reduce cohesion and hence improve flow properties, improved particle crystallinity (hence improved stability), modify particle mechanical properties. Transfer of at least one agent (to the particles to establish a stable uniform mix), ease of particle mixing, formation of stable uniform mix, prevention of particles segregation. Important delivery capabilities include particle aerodynamic properties, powder surface area, hollow volume, dissolution rate, solubility, controlled release of therapeutic agent, maintenance of the particle at the site of action, targeting different and specific regions of the airways.

The method of treating particles to engineer them with particular chemical, morphological and/or physical features, comprising the steps of:
a) processing at least one agent to form a particle;
b) treating by making available a fluid alone or in combination with at least one additive to the particle (processed or unprocessed) to promote changes in the chemical, morphological and/or physical features of the particle;
c) repeating step (b) as many times as necessary;
d) harvesting engineered particles; and repeating steps (a) to (d) as many times as necessary.

Preferably the particles to be treated is as obtained from the supplier or manufacturer without any further modifications.

Preferably the method of producing particles suitable for engineering by present method should produce particles of a narrow particle size distribution, particles of controllable size and these particles are preferably smaller than the particle size required for the intended purpose.

Suitable particles for engineering by the method of the present invention can be produced by various methods include but not limited to: spray drying, micronisation, granulation, sieving, fractioning, freezing, freeze drying, spray freezing, spray freeze drying, spray-chilling, spray congealing, spray cooling, freeze fracturing, spray freeze fracturing, emulsion solvent evaporation/extraction, coacervation, extrusion spheronisation, coating of nonpareil spheres, pelletization, wet granulation, dry granulation, crystallization or ‘MELT BACK crystallisation’.

One of the preferred methods of producing particles suitable for engineering by the present method is that of spray drying as it results in particles of controllable size with a narrow size distribution. Such a process is known to suitably produce particles from various materials (e.g. lactose).

Another preferred method of producing particles suitable for engineering by the present method is that of spray freezing, spray congealing as they produce particles of defined size and shape.

Another preferred method of producing particles suitable for engineering by the present method is that of MELT BACK crystallisation.

Preferably the particles to be treated are spherical in shape, and most preferably the particles are hollow and spherical in shape especially for inhalation purposes.

The particles to be treated can be in any state of matter. Suitable states of matter of the particle include but not limited to: solid, liquid, gas (ideal, real or mixtures thereof), vapour, supercritical fluids, solutions, suspensions, dispersions, emulsions or micro-emulsion, colloids, liquid crystals, visco-elastic, gels, waxy material, slurry, paste, semi-solid, molten, frozen states and combinations thereof.

Preferably the particles to be treated are in the solid state, liquid state (as droplets or droplets of the molten state), vapour or the frozen state.

In the method of the present invention the particles are treated with a fluid to promote the engineering of desirable chemical, morphological and/or physical features in the particles as well as transfer of at least one agent to the particle. It will be appreciated that the fluid can be made up of one or more mediums. The medium(s) of the fluid can be in different states of matter. In situations where the fluid comprises more than one medium it is preferred that the mediums of the fluid are miscible. The fluid can also comprise one or more constituents. Both constituents and mediums are agents which can or cannot be combined with an additive. The fluid may also contain agents as constituents, which are or are not present in the particle. Equally, the particle can contain agents that are or are not present as constituents of the fluid

Suitable states of matter of the fluid include but not limited to: solid, liquid, gas (ideal, real or mixtures thereof), vapour, supercritical fluids, solutions, suspensions, dispersions, emulsions or micro-emulsion, colloids, liquid crystals, visco-elastic, gels, slurry, paste, semi-solid, molten, frozen states and combinations thereof.

Preferably, the fluid is in the liquid state or vapour state. It is understood that these two states of matter may be comprised of solutions, suspensions, emulsions and colloids

Preferably the fluid used in the method of the present invention is in the liquid state. More specific examples of suitable mediums to make up the fluid include: water; hydrocarbons solvents; mineral spirit; mineral oils; halogenated solvents, such as methylene chloride and bromide, freons, bromo-chloro-methane, chloroform and carbontetrachloride; oxygenated solvents, such as ketones, ethers, esters, carboxylic acids, aldehydes, alcohols and carbonates; nitrogen containing solvents, such as amines and amides; sulphur containing hydrocarbon solvents, such as sulphoxides and sulfonates; and other hetero-atoms containing hydrocarbon solvents; mineral acids, such as sulfonic acids, sulphuric acids, phosphoric acids, nitric acids and anaesthetics such as halothane, enflurane, isoflurane, methoxyflurane, sevoflurane. Yet more specific examples are liquefied gases e.g. liquid nitrogen (boiling point −196° C.), liquid oxygen (boiling point −183° C.), liquid argon (boiling point −186° C.), chlorofluorocarbons, fluorocarbonated refrigerants (such as dichlorodifluoromethane, perfluoropropane, CF4, C2F6, C3F8, C4F8, C2F4, C3F6), hydrofluoroalkanes (such as HFA-134a, HFA-227) or any liquid medium(s) (described hereinabove) that can generate a vapour. The fluid can be used in the temperature range of −200 to 200° C.

Preferably a suitable ketone is acetone, a suitable alcohol is ethanol and a suitable liquefied gas is liquid nitrogen.

The agents comprising the particle can be completely soluble, completely insoluble or have partial solubility (anywhere in between soluble and non-soluble) in the fluid.

Preferably at least one agent of the particle should have limited solubility in the fluid.

The step of treating the particles with the fluid can, in one alternative of the present invention, involve introducing the fluid to the particle, in this instance the particle may be static or in motion. The fluid can be introduced at any rate, in any state of matter, and in bulk, as droplets, as a mist, as a fog, as a spray or combinations thereof. Alternatively, the particles are introduced to the fluid, in this instance also the fluid may be static or in motion, with such introduction being at any rate in any state of matter, and in bulk, as droplets as a mist, as a fog, as a spray or combinations thereof.

Preferably the step of treating the particles with the fluid lasts for between 1 microsecond and several hours. However, more preferably, the treating step lasts for between 1 microsecond and 60 minutes.

The step of treating the particles with the fluid can occur at the point of particle manufacture, wherein for example, the particles are fully or partly formed and then treated with the fluid, alternatively the particles can be engineered in the fluid as the particle crystallizes or forms within the fluid as in melt-back crystallisation.

Before, during or after treating particles with the fluid, additives can be applied to the particle, fluid or both in order to engineer the required features of a particle for the particular task.

Additives generally include but not limited to such factors as: heat (directly or resulting from the application of laser energy or microwaves), moisture, radiation (laser light, microwaves), pressure, vacuum, shear forces, magnetic forces, vibration, systems of agitation, stirring, rotation, tumbling, vortexing, centrifuging, masticating, ultra-sound waves or extruding and electrical, although any factor or combination of factors that favour changes in the chemical, morphological and/or physical features of a particle are desirable. For example the use of stirring and heat increases the speed and extent of particle growth, hair growth and pore size and uniformity in the change of the particle properties.

It is also further appreciated that before, during or after treating the particles with the fluid at least one further agent can be applied (or added) to the particle, fluid or both in order to engineer the required features of a particle for the particular task. For example agents incorporated (or added) to the particle and agents added to the fluid aid the formation and growth of hairs, impart plastic behaviour to the particle, maintain the spherical shape of the particle and transfer agents from the fluid onto the particle.

Preferably the agent(s) can be either a therapeutic agent, prophylactic agent, diagnostic agent or an excipient. It is also appreciated that more than one of such agents may be used in combination to create the engineered particles of the present invention. Other materials commonly used in pharmaceutical compositions, such as diluents, flavourants, fragrances, dyes, nutrients and sweeteners are also considered as possible agents within the understanding of the present invention.

Suitable nutrients include: retinoids such as all-cis retinoic acid, 13-trans retinoic acid and other vitamin A and beta carotene derivatives, vitamins D,E,K and water insoluble precursors and derivatives thereof.

The therapeutic agents, prophylactic agents and diagnostic agents of the present invention are preferably taken from the group comprising: peptides, proteins, organic substances, inorganic substances, pro-drugs, antigens and hormones.

More specific examples of agents that can be treated under the present invention include: corticosteroids; anti-inflammatories such as beclomethasone, betamethasone, fluticasone, flunisolide, budesonide, dexamethasone, tipredane, triamcinolone acetonide; anti-tussives such as noscarpine; and bronchodilators such as ephedrine, adrenaline, fenoterol, formoterol, isoprenaline, metaproterenol, phenylephrine, phenyl propanolamine, pirbuterol, reproterol, rimiterol, salbutamol, salmeterol, formoterol, terbutaline, isoetharine, tulobuterol, orciprenaline and (−)-4-amino-3,5-dichloro-α[[[6-[2-(2 pyridinyl)ethoxy}hexyl]amino]methyl]benzenemethanol,

Further specific examples of suitable agents include: the diuretic amiloride; anticholinergics such as ipratropium, ipatropium bromide, atropine, oxitropium and oxitropium bromide; hormones such as cortisone, hydrocortisone and prednisolone; and xanthines such as aminophylline, choline theophyllinate, lysine theophyllinate and theophylline.

Yet further specific examples of suitable agents include: analgesics such as codeine, dihydromorphine, ergotamine, fentanyl and morphine; diltiazem which is an anginal preparation; antiallergics such as cromoglycate, ketotifen and nedocromyi; anti-infectives such as cephalosporin, penicillins, streptomycin, sulphonamides, tetracyclines and pentamidines; and the anti-histamine methapyrilene.

Yet further specific examples still include: anti-neoplastic agents like bleomycin, carboplatin, methotrexate and adriamycin; amphotericin B; anti-tuberculous agents such as isoniazide and ethanbutol. Therapeutic proteins and peptides (e.g. insulin and glucagon, prostaglandins and leukotrienes) and their activators and inhibitors including prostacyclin (epoprostanol), and prostaglandins E, and E2 are also considered to make suitable substances for treatment using the method of the present invention.

It will be appreciated to the artisan that, where appropriate, the above listed therapeutic agents may be used in the form of salts (e.g. as alkali metal or amine salts or as acid addition salts) or as esters (e.g. lower alkyl esters) or as solvates (e.g. hydrates) to optimise the activity and/or stability of the therapeutic agent.

Preferably, where the agent is a therapeutic agent it will either be an anti-inflammatory drug or a bronchodilator. More specifically the preferred therapeutic agents of the present invention are beclomethasone dipropionate, salbutamol sulphate and fluticasone propionate.

Preferably, when the excipient is used on its own to produce particles and not in combination with any other type of substance (i.e. therapeutic agents, prophylactic agents and diagnostic agents) such excipients are sugars, preferably taken from the group comprising: monosaccharide, disaccharide, polysaccharide and sugar alcohols such as sorbitol, mannitol, maltitol. Further preferably the excipient is lactose.

It is also appreciated that more than one of the above agents may be used in combination to produce the particles of the present invention. Suitable combinations comprise a short acting β₂ agonist and an antimuscarinic, typically salbutamol and ipatropium bromide; or fenoterol and ipatropium bromide. Alternatively the combination of a short acting β₂ agonist and a corticosteroid in the form of salbutamol and beclomethasone is advantageous. A further alternative is the combination of a long acting β₂ agonist and a corticosteroid, typically salmeterol and fluticasone; or eformoterol and budesonide.

As discussed above, the combination of one or more therapeutic agent, prophylactic or diagnostic agent (as listed above) with one or more pharmaceutical excipients is also considered desirable within the present invention. The excipients suitable to be used in combination with therapeutic agent are not necessarily the same as those that are appropriate when a particle is produced from an excipient alone.

The presence of an excipient in combination with a therapeutic agent can facilitate a retarded, controlled, sustained or targeted release of the therapeutic, prophylactic or diagnostic agent. According to the present invention excipients can act to regulate the release, such excipients are preferably either non biodegradable, biodegradable or bioerodible polymers.

More specifically, suitable polymers include but not limited to: cyclodextrins and derivatives thereof, sodium caseinate, dipalmitoyl phosphatidyl choline (DPPC), human serum albumin, phospholipids, cellulose acetate phthalate, hydroxypropyl methylcellulose phthalate, ethyl cellulose, hydroxypropyl methyl cellulose, hydroxyethyl cellulose, hydroxypropyl cellulose, ethyl hydroxyethyl cellulose, carboxymethyl cellulose, methyl cellulose, cellulose acetate butyrate, poloxamer, poly(lactic acid), poly(lactic-co-glycolic acid), poly(lactide)s, poly(glycolide)s, poly(lactide-coglycolide)s, poly(p-dioxanones), poly(caprolactone), polycarbonates, polyamides, polyanhydrides, poly(alkylene alkylate)s, polyamino acids, polyhydroxyalkanoates, polypropylenefumarates, polyorthoesters, polyacetals, polyacrylamides, polycyanoacrylates, polyalkylcyanoacrylates, polymetha polyphosphate esters, polyphosphazene, polyurethanes, polyacrylates, polymethacrylate, poly(methyl methacrylate), poly(hydroxy ethyl methacrylate-co methyl methacrylate), carbopol 934, ethylene-vinyl acetate and other acyl substituted cellulose acetates and derivatives thereof, polystyrenes, polyvinyl alcohol, polyvinyl pyrrolidone, polyvinyl chloride, polyvinyl fluoride, poly(vinylimidazole), chlorosulphonated polyolefins, polyethylene, polyethylene glycols, polypropylene, polyethylene oxide, copolymers and blends thereof.

Preferably, the selected polymer is biocompatible in that it degrades or erode in-vivo to form non-toxic small molecules. More preferably, the biocompatible polymer is pharmaceutically acceptable for delivery to the respiratory tract. Even more preferably, the polymer is both pharmaceutically acceptable to the lung and has therapeutic properties.

In another preferred aspect, the selected polymer imparts to the resulting particles a plastic nature. Examples of polymers that impart a plastic nature to the particles are Polyvinyl alcohol, Polyvinylpyrrolidorie and polyethylene glycol (PEG).

In another preferred aspect, agents other than the polymer can impart to the particle a plastic nature.

In another preferred aspect, the fluid (with or without additives) can impart to the particle a plastic nature directly or by the transfer of agents with a plastic nature to the particles.

In another preferred aspect, the selected agent may impart to the particle a brittle nature.

In another preferred aspect, the fluid (with or without additives) can impart to the particle a brittle nature.

In view of the above comments, preferable excipients for use in combination with one or more therapeutic, prophylactic or diagnostic agent are cellulose acetate phthalate, hydroxypropyl cellulose acetate phthalate, polymeric drugs and genetically engineered polymers.

The agent or agents to be treated might contain one or more stabilisers to protect the therapeutic agent from degradation and maintain the biological activity. The term stabilisers as described herein means any agent which binds or interacts in a covalent or a non-covalent manner with the therapeutic, prophylactic, diagnostic agent or excipient. Suitable stabilisers that can be used in the present invention will be appreciated by the skilled man (see for instance U.S. Pat. Nos. 5,716,644; 5,674534; 5,654,010, 5,711,968; 6,284,283). However, preferred stabilising agents include: sucrose, trehalose, polyvinyl pyrrolidone and dextran.

It is appreciated that the agent in the particles and/or in the fluid can have preservative, antiseptic, disinfection and/or sterilisation properties. When these agents are combined with additive such as heat or radiation increases the efficiency of preservation, antiseptic, disinfection and/or sterilisation effects.

Suitable preservative, antiseptics, disinfectants and sterilising agents include but are not limited to: phenolics (such as: phenol, cresols, xylenols), halogenated phenolics (such as, chlorocresol, chloroxylenol, hexachlorophene, triclosan), alcohols (such as, ethanol, benzyl alcohol, bronopol, phenoxy-ethanol), aldehydes (such as, formaldehyde, glutaraldehyde), organic acids and their ester, quaternary ammonium compounds (such as, cetrimide, benzalkonium chloride), biguanides (such as chlorhexidine, polyhexamethylene biguanide), amidines (such as, propamidine, dibromopropamidine), halogens and their compounds (such as, hypochlorous acid, Eusol, Chloronated Soda solution, chloramines T, halozone, potassium iodide, iodophores, betadine), Metal ions (such as, mercury, silver, aluminium, phenylmercuric nitrate and acetate, thiomersal), acridines (such as, aminacrine hydrochloride), gases (such as ethylene oxide, formaldehyde, β-propiolactone, propylene oxide, methyl bromide, gas plasmas in combination with heat or radiation).

Wherein the fluid itself either in liquid or vapour state has preservative, antiseptic, disinfectants, sterilising properties in addition to architecturing the particles. The liquid sterilising agent is an alcohol in this case ethanol and the vapour state is steam.

In a further aspect of this embodiment the material of the particle is softened by treating with the fluid, the frequency and energy of the radiation source (laser light or microwaves) may be matched with the physico-chemical properties of the particle such that the radiation causes fracturing, dimpling of the particle or if the particle is hollow forming holes within the particle or ablating the particles.

It will be appreciated that the above discussed additives can be utilised at any stage or stages a) to d) of above described method.

It will be appreciated that the treatment of a particle with a particular selection of agents and environmental additives will result in the formation of a particle with a particular set of chemical, morphological and/or physical features. Some of the features may be desirable for the chosen task of the engineered particle, whereas some features may not. In such circumstances it is appreciated that a pre-treated particle, produced by the treatment with a first set of fluids, mediums, agents and additives, can be subjected to further treatment with another particular selection of fluids, mediums, agents and additives (such treatments can be repetitions of the earlier treatment or alternative treatments).

Important morphological features that can be engineered on particles subjected to the present invention include: hairs; spongy-like formations; porous; surface dimpling, particle shape, particle surface texture, transfer of at least one agent to the particle and combinations thereof. More specifically for pores, the size, shape and number of the pores is important. The term hair, used throughout this specification is considered to include any type of projection present on the surface of a particle. Such projections can be of any shape (e.g., needle, plates, blade, fluffy), size, texture, density and have any mechanical property, (e.g., elastic, brittle, plastic, glassy). Preferably any engineered hairs are within the range of 0.001 micrometers to 5000 micrometers in length.

Important chemical and physical features that can be engineered to the particle subjected to the present invention include: particle size, density, specific surface area and surface texture; mechanical properties such as friability, tensile strength, elastic, brittle, plastic, glassy and rubbery states; polymorphism or crystallinity; solubility and dissolution rate; aerodynamic properties, hygroscopicity, cohesiveness, particle hollow volume, ability of the particles of the present invention to improves blend homogeneity, improves the aerosolization and deposition of highly cohesive and poor flowing particles, the result of transfer of at least one agent.

A suitable size for particles is considered to be between 0.05 and 4000 μm in diameter, furthermore for particles intended for inhalation or as carrier for inhalation are of a preferred particle size between 0.05 and 200 micrometers. The most preferred particle size for inhalation or as carrier for inhalation is between 0.5 and 60 micrometers.

It will become apparent to the artisan, herein below, that one or more features of the particle can be engineered whilst maintaining the other features

The particle size can be determined by conventional particle size measuring techniques known to those skilled in the art, such as, laser diffraction, photon-correlation spectroscopy, sedimentation, field-flow fractionation, disc centrifugation or electrical sensing zone; the most preferable being laser diffraction.

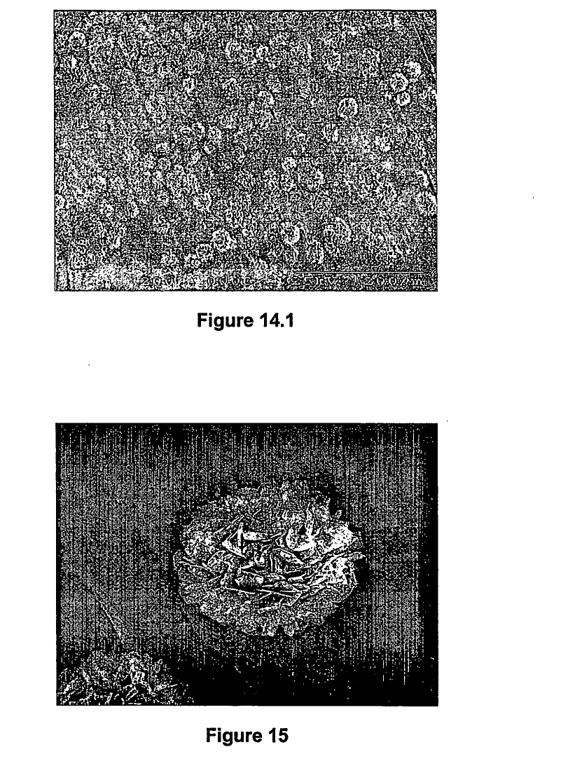

In one aspect of the second embodiment, the process of the present invention reduces the particle size by over 1000% whilst maintaining the original shape and the particles are mono-disperse. In FIG. 14 the starting particle is as small as 3 mm in diameter whilst the final particles have a maximum diameter of 30 micrometers (FIG. 14.1). This represents a phenomenal reduction in particle size whist the original shape of the particles is maintained, in addition, hairs are advantageously produced.

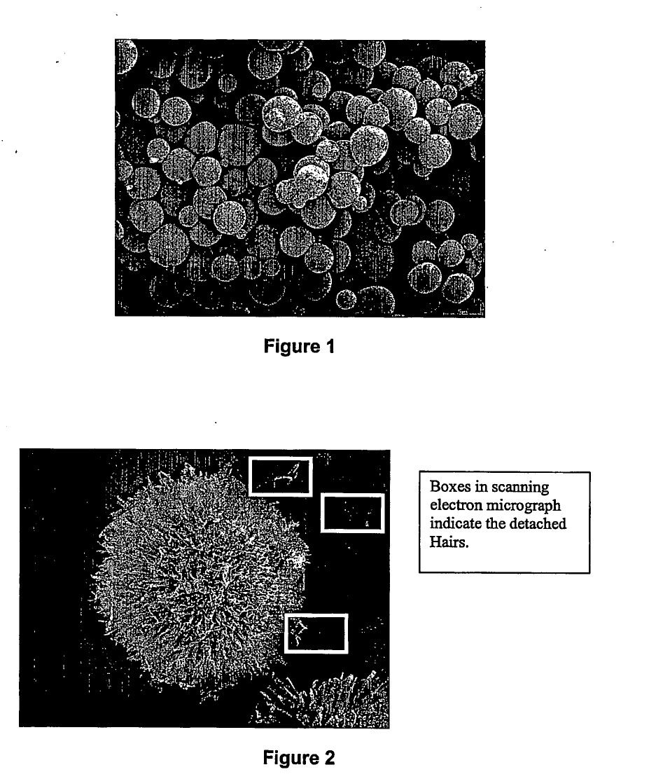

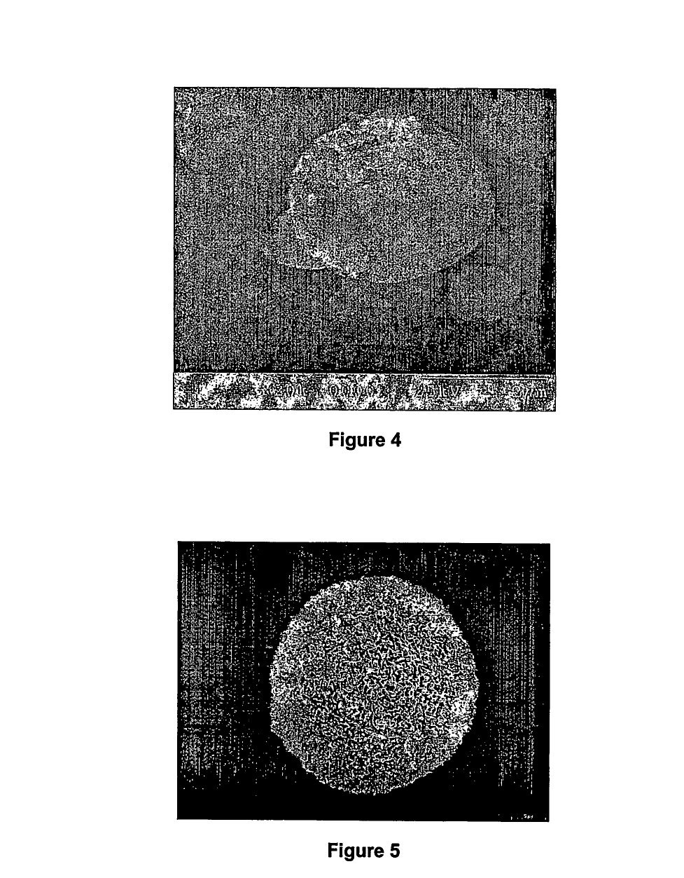

The process of the present invention can maintain the particle size (Examples 2, 4,9 and 11), compare FIGS. 4, 8b, 8c, 20, 21 with that of the starting particles of FIGS. 1, 8a and 19.

Preferably the process of the present invention enables controlled growth in the particle size. Compare the starting particle size of FIGS. 1, 10 and 12 with the corresponding treated particles of FIGS. 2, 5, 6, 7, 11,11.1,13,16 (Examples 1, 2, 3, 5, 6 and 8). From FIG. 7 it is clear that the particle grows to at least 70 micrometers.

To exemplify that the present invention can control the size of the resultant particles the following table list examples of Figures achieving a certain particle size.

| Particle Size | Figures in which the particles |
|---|---|
| (micrometers) | are of this range |
| 10 or below | 4, 8a, 8b, 11.1 and 18 |
| 20 | 6 and 16 |
| 30 | 5, 11, 14.1 |
| 60 | 13 |
| 70 | 2 and 7 |
| 80 | 9 |

Thus with this invention it is possible to control the particle size and produce particles much greater than 80 micrometers by manipulating at least the treatment time, treatment temperature and treatment conditions.

It should be apparent to the skilled artisan that the growth and size of the resultant particles is dependent upon the size of the starting particle. Hence if a large particle is required the preferred starting particle size should also be large.

In the current invention the particle size can be manipulated over a size range of 5 mm, the preferred particle size control range is 200 micrometers and the most preferably particle size control range is 80 micrometers

In another aspect of the second embodiment, the process of the present invention preferably enables the alteration of the particle density (see example 2, FIGS. 5,6,11.1,15,18 and 20 by altering treatment conditions or mass transfer FIG. 16).

The powder was carefully poured into a 50 ml volumetric cylinder and the particles bulk density was calculated by dividing the weight of powder (gm) by the volume occupied by the powder bed (ml). The results are shown below.

| | Bulk density | Example in |
|---|---|---|
| Material | (gm/ml) | figure no. |
| Spray dried lactose | 0.276 | 1 |
| Liquid vapour introduced to | 0.240 | 8c |
| the lactose particles | | |
| Lactose particles added to the | 0.182 | 6 |
| liquid fluid | | |

It is clear that by changing the method in which the particle is engineered it is possible to manipulate the density

In yet another aspect of the second embodiment, the process of the present invention preferably reduces the cohesiveness, adhesiveness and enables improvement in the powder flow (Examples 14 and 16). Treatment with the vapour of low temperature liquid gases and or immersion in low temperature liquid gases were extremely effective in reducing cohesion between particles (Example 13). To those skilled in the art, it should be apparent that particles with minimal adhesion and cohesion are excellent for dry powder inhalers.

In a further aspect of the second embodiment, the process of the present invention preferably maintains the particle shape (see all the examples and all the figures).

In the background of the invention, the importance of spherical shaped particles was described in the examples presented, the starting particles and final particles were spherical, thus the method of the invention maintains the preferred spherical shape.

In a still further aspect of the second embodiment, the process of the present invention preferably form and/or modify hairs on the particles. The process of the present invention preferably controls the size of the hairs to produce nano-sized projections (FIGS. 4,5,6, 8c), micron sized projections (FIGS. 2,7,11,11.1,15) and projections that are larger than the particle core (FIG. 7).

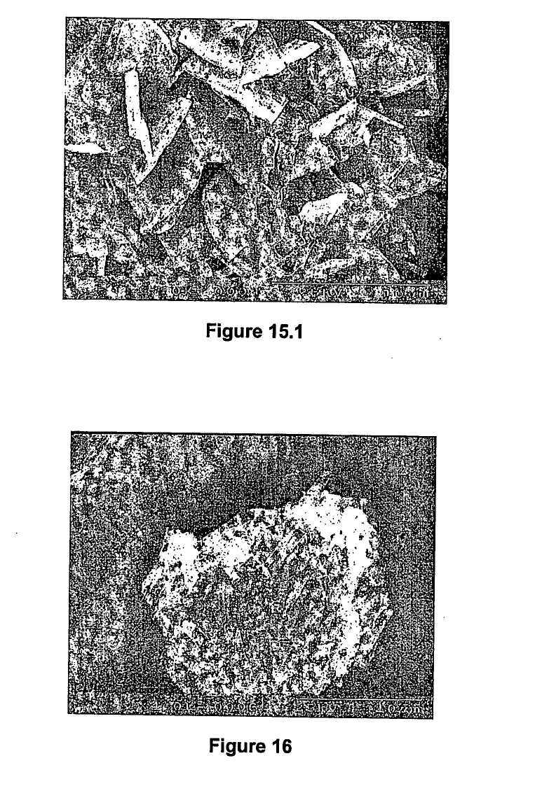

Further, the process of the present invention also preferably controls hair shape to obtain, for example, crystalline hairs (FIGS. 2 and 7), fluffy hairs (FIG. 11.1), blade-like hairs (FIG. 15), hairs tangential to the surface of the particle (FIG. 16) and plate-like hairs.

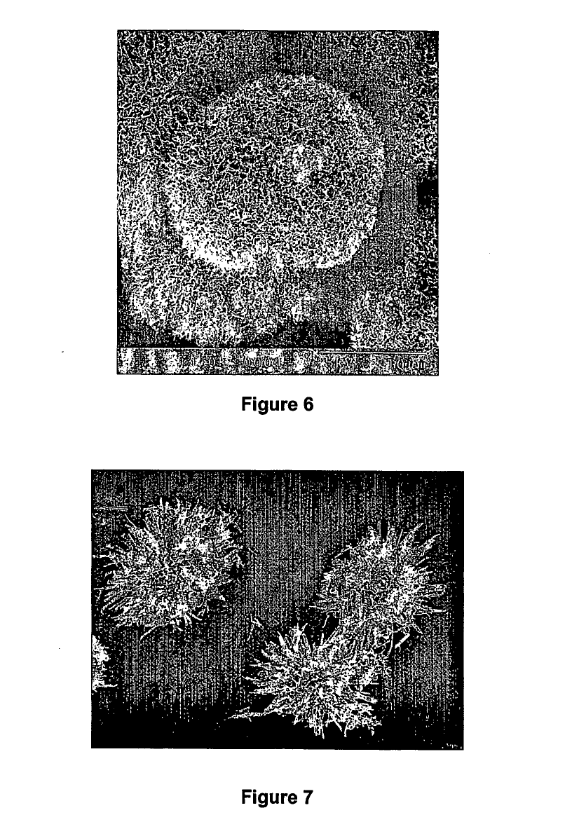

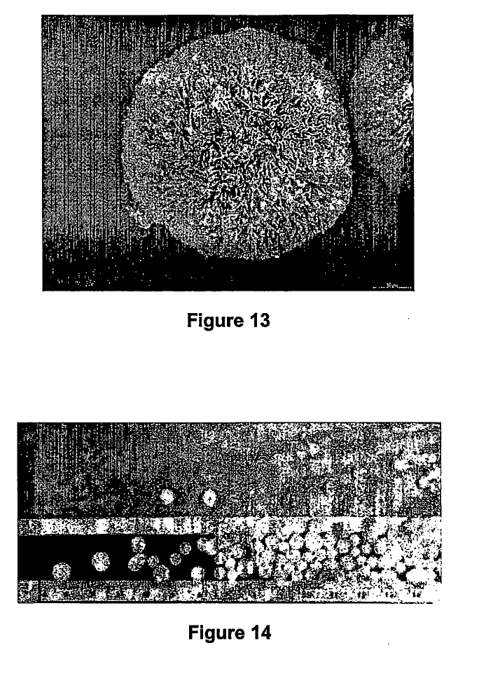

Furthermore, the process of the present invention also preferably controls the nature of hairs, for example, in producing crystalline, elastic and brittle hairs which can detach from the particle (FIGS. 2 and 7) or hairs that exhibit a plastic nature resulting from the incorporation of a plastically deforming material such as PVP (FIG. 13) or PVA (FIG. 11.1) into the particle or the use of an additive such as heat to induce a material that is normally brittle to produce plastically deforming hairs (FIG. 6).

The hairs formed on the particle can be combinations of hairs produced directly from the agent(s) of the particle (FIG. 2) or may be the result of the transfer of at least one agent to the particle from the fluid (FIGS. 16 and 20).

In another aspect of the second embodiment, the process of the present invention preferably changes the mechanical properties of the particles and hairs.

The process of the present invention can preferably induce a brittle nature to the particles and the hairs (FIGS. 2 and 7). Treating the particles with low temperature liquid gases (such as liquid Nitrogen as in Example 13) are known to induce a brittle nature to the particle treated.

Furthermore, the process of the present invention can preferably induce a tendency of the particles to deform plastically (FIGS. 11, 11.1 and 13). This change in particle mechanical properties results from the nature of the agents of the particle, nature of agents introduced to the particle and/or treatment conditions such as the use of heat.

The advantages of using liquid nitrogen or liquid nitrogen vapour and their combinations is that it is applicable to both water soluble and water insoluble agents. With liquid nitrogen, considerably greater evaporation rates, at considerably lower temperatures, is obtainable. Consequently the low temperature is less likely to affect heat sensitive materials and is therefore applicable to proteins, peptides, macro-molecules and heat sensitive agents. The high evaporation rate gives a high specific area of contact between liquid nitrogen vapour and the particles thereby reducing the treatment and drying time. Vapours are also known to reduce the electrostatic charges between the particles reducing cohesiveness, adhesion and improving flow properties of the powder as detailed in the second embodiment. Liquid nitrogen is also environmentally friendly. Heat may impart to the particle a plastic nature.

In another aspect of the second embodiment, the process of the present invention, further and preferably modifies the surface texture for example from smooth surfaces to increasing degrees of surface roughness, All figures except FIGS. 1, 2.1, 8a, 10, 12 and 19, Examples 1 to 12).

In another aspect of the second embodiment, the process of the present invention preferably form or modify pores in number, shape and size (FIGS. 4,6, 8c [golf ball-like], 11.1, 17, 18, 20 and 21 see Examples 0.2, 3, 4, 5, 9, 10, 11 and 12).

In another aspect of the second embodiment, the process of the present invention preferably maintains or increases particle hollow volume (FIGS. 2, 5, 6, 11, 11.1 and 13, see Examples 1, 2,3, 5 and 6).

In another aspect of the second embodiment, the process of the present invention preferably gives the operator control of the specific surface area of the particle (see example 7).

It is known that the specific surface area of a spherical particle is the ratio of the surface area of the sphere (4πr²) divided by the volume of said sphere (4πr³/3)

$$Specific \text{-} surface \text{-} area = \frac{Surface \text{-} area}{Volume} = \frac{4 π {r}^{2}}{\frac{4}{3} π {r}^{3}} = \frac{3}{r}$$

From the above it is clear that the specific surface area increases as the radius of the particle decreases. Hence control of the particle size gives the skilled artisan control of the specific surface area and since the particle size can be controlled by the present invention then also the specific surface area can also be controlled as shown below.

The following tables list the radius, corresponding volume, surface area and specific surface area of spherical particles produced according to this invention. It is evident that the starting and final particles are spherical and that the starting particles have a minimum diameter of 3 mm (see FIG. 14, with radius of 1.5 mm) whilst the final particles have an diameter of 30 micrometers (see FIG. 14.1. hence the radius is 15 micrometers which is 0.015 mm).

| | | | Surface area | Specific Surface |
|---|---|---|---|---|
| | Radius(mm) | Volume (mm³) | (mm²) | area (1/mm) |
| Starting | 1.5 | 14.14 | 28.27 | 1.999 |
| particle | | | | |
| Final | 0.015 | 0.0000141 | 0.00282 | 200 |
| particle | | | | |

It is clear that the process of this invention enables an increase in the specific surface area of the particles.

Apart from increasing the specific surface area of the particle by reducing the particle size, the hairs and pores formed on the particle further increases the surface area of the particle thereby further increasing the specific surface area of the particle.

Equally consider example 0.3. where the starting particle size of spray dried lactose is 2 micrometers (FIG. 1 this equates to a radius of 1 micrometer which is 0.001 mm), after treatment with the process of the current invention the final particle size is at least 20 micrometers (FIG. 6, equating to a radius of at least 10 micrometer which is 0.01 mm). It is also clear that both starting and final particles are spherical in shape, hence the above equation is applicable. Putting this data in a similar table as above.

| | | | Surface area | Specific Surface |
|---|---|---|---|---|
| | Radius (mm) | Volume (mm³) | (mm²) | area (1/mm) |
| Starting | 0.001 | 4.2 × 10⁻⁹ | 1.3 × 10⁻⁵ | 3100 |
| particle | | | | |
| Final | 0.01 | 4.2 × 10⁻⁶ | 1.3 × 10⁻³ | 310 |
| particle | | | | |

It is clear that the process of this invention enables a decrease in the specific surface area of the particles.

Hence the process of the present invention enables the skilled artisan to manipulate the specific surface area as required.

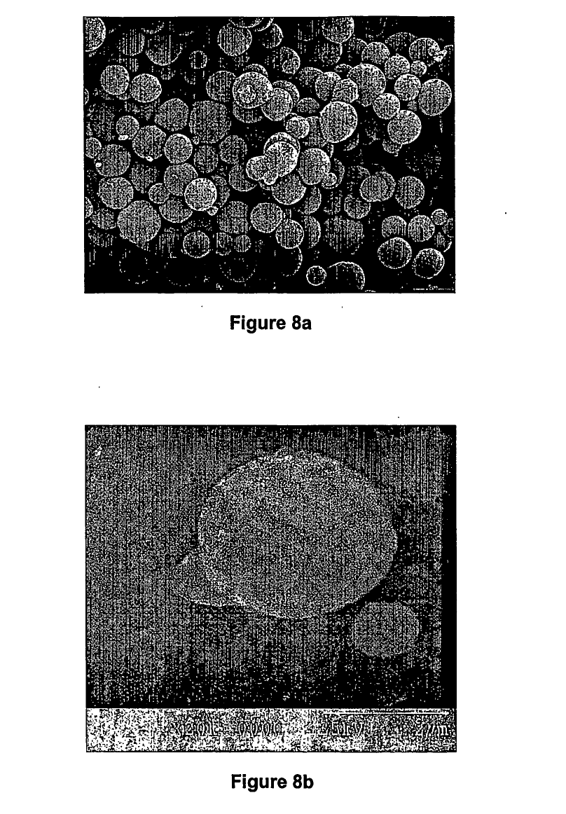

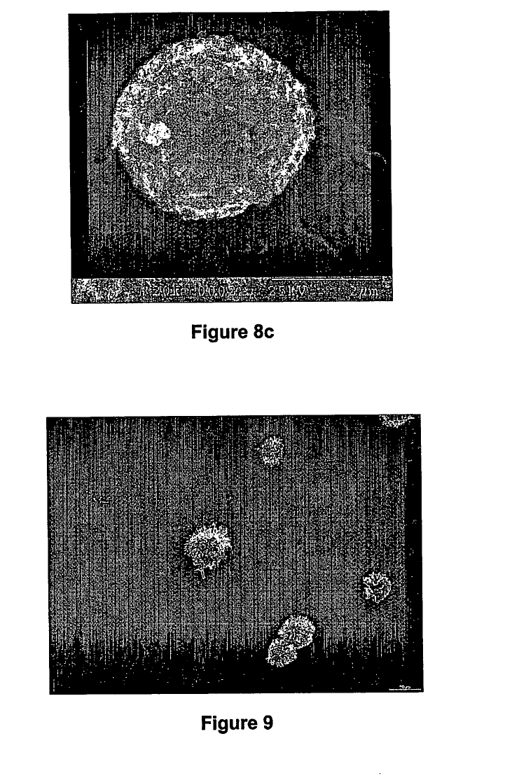

In another aspect of the second embodiment, the process of the present invention preferably forms or modify dimple on the particle (FIGS. 4, 8c see Examples 2 and 4).

In another aspect of the second embodiment, the process of the present invention preferably causes spongy-like formations (FIGS. 5, 6, 11, 11.1 and 20 see Examples 2, 3, 5 and 11).

In another aspect of the second embodiment, the process of the present invention preferably modifies the aerodynamic properties of the particle (see example 15). From FIG. 23.3 it is clear that hairy particles of the present invention, with geometric mean diameters as large as 50 micrometer deposit in the lower stage of the twin stage impinger and consequently such particles should deposit in deep lung. This in itself is contrary to the prior art, where particles with geometric mean diameter of 5 micrometers or less deposit in deep lung.

In another aspect of the second embodiment, the process of the present invention preferably transfers an ultra-fine agent to another particle of the same or larger size to form a stable mix (FIGS. 16 and 20 see examples 8 and 11).

In another aspect of the second embodiment, more preferably, the process of the present invention enables the control of combinations of the above, i.e. particle size, hairs, surface area, hollow volume, particle density, powder flow and transfer of an agent (FIG. 20 and example 11).

In another aspect of the second embodiment, the particles of the present invention preferably improves blend homogeneity composed of engineered particles of this invention and low dose drug (see example 16).

In another aspect of the second embodiment, the particles of the present invention preferably improves the aerosolization and deposition of highly cohesive and poor flowing particles, an example given for clarity is spray dried lactose (i.e. engineered particles mixed with spray dried lactose and aerosolised into a twin stage impinger see example 15).

A third embodiment of the present invention relates to a particular family of therapeutic agent delivery particles (i.e. carrier particles) for the delivery of drug via inhalation into the lungs, which can be produced by the method of this invention. The carrier particles of this embodiment have specific engineered features which make them better suited for the delivery of drugs deep into the lungs. The present invention provides carrier particles with improved lung deposition of therapeutic agents. These improved carriers are low in density and tend to have non-smooth (e.g. containing pores or hairs according to the invention) surfaces. As was discussed earlier in this document, it is appreciated that both of these characteristics run contrary to the accepted prior art.

The carrier particles of the present invention is preferred to have hairs, more preferably the carrier particle should be hairy and porous or hairy and low density and most preferred the carrier particle is hairy, porous and of low density. It is further preferred that the carrier particle has good aerodynamic properties and this may be the result of manipulating combinations of the particles hairy, porous and low density nature.

From the second embodiment it should be apparent to the skilled artisan that this present invention enables the production and manipulation of particle hairs, pores, hollow volume and density. In addition, the present invention also reduces cohesiveness of the particles, improves the flow of the particles, improves aerosolization of the particles (by reducing both cohesive and adhesive forces) and good particle deposition to the lower stage of the twin stage impinger (See example 15). All the improvements detailed above, consequently improve the aerodynamic properties of the particles. The literature suggest that the effective cut-off diameter for a twin stage impinger at 60 litres per minute is 6.3 to 6.4 micrometers (Hallworth. G. W. & Westmoreland, D. G., 1987, The twin impinger: a simple device for assessing the delivery of drugs from metered dose pressurized aerosol inhalers, J. Pharm., Pharmacol. 39, 966-972 and Miller et al, 1992, Assessment of the twin impinger for size measurement of metered dose inhaler spray. Pharm. Res., 9, 1123-1127). However, from example 15 and FIG. 23.3 the engineered particles of this invention, despite it's larger size (i.e. 40-50 micrometer, FIG. 23.3) it still deposits in the lower stage of the twin stage impinger suggesting that it's aerodynamic diameter is less than 6.4 micrometer and must be a consequence of the hairs, pores, hollow volume and low density of the particle. Conventional carriers particles remain in the inhaler device and or deposit in the mouth or on back of the throat hence they have a short time of flight. Whereas the engineered carriers of this invention must have travelled a long distance to reach the lower stage of the twin stage impinger suggesting that they have a much longer time of flight than conventional carrier particles. The long flight time of the engineered carrier particles mean that these particles can carry cohesive drugs into deep lung. Hence the adhesion and cohesion problems that are normally associated and are burdens for traditional carrier particles are of no consequence with the carrier of the current invention. These particles are consequently desirable for deep lung penetration.

The present invention enables the production and manipulation of hairs. The presence of hairs on the particles gives the particle many attributes some of which are detailed below. The hairs maintain the stability and content uniformity of the mix (example 17). The hairs are part of the particles and can act as a ternary component that minimises contact between the carrier core and therapeutic agent particle. In addition the hairs have a dynamic element whereby oscillation of the hairs improves detachment of adhered mono-disperse or poly-disperse therapeutic agent particles from the carrier particles. A slight oscillation may be sufficient to detach the hairs from the particle during the time of flight (See FIG. 2 which depicts some detached hairs before aerosolization). Some of the remaining hairs attached to the particle and the particle itself, at impact, fragment ejecting considerable quantities of hairs and particle fragments over an area substantially larger than the impact site (See FIGS. 23.3, 23.4 and example 15) giving a “cluster-bomb” like effect. This consequent increase in lung contact area is pharmacologically important in that a larger area of the lung is treated at any one time compared to conventional particle thus making these particles more economic as a larger area of the lung is treated with minimal amount of the drug. As a result pharmacological bioequivalence is achieved using a smaller amount of engineered drug particles compared to the larger amount of drug used in the conventional inhaler systems. Giving considerable cost savings especially in cases where the drug is expensive whilst minimising unwanted side effects. The increased surface area conferred to the particle by the hairs automatically improves the aerodynamic properties of the particle as well allowing more therapeutic agent particles to be adhered to one carrier particle, thereby reducing the carrier particle: therapeutic agent particle ratio. Further, the hairs can be produced of bioadhesive agents which at the point of carrier impaction allow the hairs to act as grappling hooks achoring the carrier particle to the impact site. Furthermore, the mechanical properties (elastic/brittle/plactic behaviour) of the hairs can be manipulated to prevent bouncing of the particles in the lung epithelia, this bouncing effect is a source of expiration of small particles from the lungs.

The present invention, preferably maintains the spherical shape of the carrier particle and as detailed, above in the background to the invention and summary of the invention, spherical particles gives the particles many attributes some of which are detailed below. Spherical particles are easier to mix than any other shape. Spherical particles have optimal flow properties due to minimal inter-particulate contact and minimizes segregation. The improved flow properties resulting from the spherical shape of the particles enable easier and total aerosolization of the powder.

The present invention, further, preferably enables control of the particle size (whether by shifting over-size particles to undersize particles or controlled growth of the particles to the required particle size), density (by transferring agents to the particle to increase the density or manipulating the treatment conditions to decrease density or combinations thereof), the pores of the particle and the hollow volume of the particle all of which alters the aerodynamic properties of the resultant particle. The carrier particles of the third embodiment are preferred to be of low density and preferable aerodynamic diameter. The latter is achieved by controlling and or manipulating the particle size, particle hollow volume and particle pores according to the equation dₐ=d_{g}(ρₚ/ρ₀χ)^{0.5}, the skilled artisan should appreciate that modification of these parameters also modifies the particle density. The low density carrier with favourable aerodynamic diameter permits easy and total aerosolization of the formulation, reduces cohesiveness of the particles, facilitates a long flight time of the carrier that in turn allows more time for the therapeutic agent particles to detach. The oscillation of hairs on such low density, aerodynamically favourable particles promote further detachment of therapeutic agent particles from the carrier. Those therapeutic agents particles which do not detach from the carrier particles are carried to deep lung to the impact site of the light, low density carrier particle where the bioadhesive and anchoring functions of the hairs retain the therapeutic agent at the lung epithelia for sufficient a time to enable therapeutic agent transfer to the lungs. The carrier particle of the third embodiment consequently delivers more therapeutic agent to the site of action in deep lung.

Hence, for the carrier of the third embodiment, the adhesion problems associated with conventional inhaler devices are of no consequence as the carrier of the third embodiment travels to deep lung. Hence the therapeutic agents may coat the hairs of the particle, coat the particle (as described in the fourth embodiment or otherwise), be strongly or weakly adhered to the carrier particle. Further the carrier particles of the third embodiment can thus be used to carry conventionally prepared (i.e. milling, spray drying and crystallisation) therapeutic agents particles or therapeutic agent particles prepared according to the embodiments of this invention. Furthermore, the amount of carrier travelling to deep lung is reduced as increased surface area imparted to the carrier particle by the hairs increases the drug loading per individual carrier particle.

Advantageously, the engineered carrier may be composed of 100% therapeutic agent, thus allowing the therapeutic agent to be delivered on its own or act as a carrier for one or more therapeutic agent particles The carried therapeutic agent particles can be traditionally prepared or engineered according to the current invention.

The fourth embodiment of the present invention is the application of the method of this invention to a process of micronising and mixing in one step that by passes the limitations of the current state of the art in micronising and mixing. This embodiment relates to a process of micronising without using conventional milling, spray drying or conventional crystallisation techniques to produce ultra fine particles. These ultra-fine particles are attached to particles of the same size or of larger size to form a stable uniform mix avoiding segregation. Another aspect of fourth embodiment is the introduction of the particles into bulk fluid.

When a potent therapeutic agent is used in a mix, the amount of therapeutic agent used is small and to ensure a uniform mix it is necessary to increase the number of therapeutic agent particles per sample or dose. To do this it is necessary to use a smaller therapeutic agent particle size, however, producing such very fine powder is difficult and often attended by severe aggregation (using conventional milling, spray drying or crystallisation techniques) thus defeating the object of size reduction in the mixing process. When the proportion of therapeutic agent is extremely small and finally presented in a small dose unit, physical dry mixing of solids will fail to produce an adequate dispersion of therapeutic agent within the formulation. To avoid the above drawbacks, the fourth embodiment of the current invention adopts an efficient and reproducible strategy in which the therapeutic agent is maintained in a liquid. The resulting therapeutic agent-liquid mix is reduced in size by atomisation to form a fine mist. This fine mist contains individual liquid droplets whose size is much smaller than that obtainable by conventional milling such as micronisation and spray-drying. Furthermore these liquid droplets are uniform in size and therapeutic agent content. Using the right atomisation protocols, the size of the liquid droplets can be arranged to be several orders of magnitude smaller than that of carrier particle with which it is mixed. Mixing of the liquid droplets and carrier results in an efficient, uniform and stable mix. The therapeutic agent adhered to the particle is uniformly distributed and smaller in size than the carrier. The therapeutic agent particles can consequently travel with the low density engineered carrier, of the third embodiment, to the deep lung. The small size of the therapeutic agent particle enables fast dissolution and transport in lung epithelia. Hence, the problems of adhesion and cohesion encountered in traditional dry powder inhalers is of no consequence with this invention which is in direct contradiction to the current state of the art.

Advantageously, one or more agents (one or more of which may be therapeutic), carried in a liquid or vapour-loaded state can be transferred to the particle. This liquid state, vapour-loaded state and transferred agent corrects surface defects, restructures the surface of the particles which in turn reduces the cohesiveness of the particles, alters the particle density, particle size and thus their low properties. The transferred agent is uniformly distributed to the particles forming a stable and homogeneous mix. The preferred vapour-loaded states for small quantities of agent transfer include mist, droplets, foam, spray, steam, fog or vapour. The vapour-loaded transferred state is more efficient and effective than conventional methods of mixing dry micronised powders.

Further the particles adhered to the carrier can be present on the carrier as discrete, discontinuous or continuous particles or films. Apart from transferring agent particles to the carrier, the method of transfer using the current invention can be manipulated to also change at least one or combinations of one or more morphological, chemical or physical features of the particle and/or, transferred agent according the embodiments of this invention. In addition the change of at least one or combinations of one or more morphological, chemical or physical features of the particle can be manipulated to occur before, during or after the transfer of the agent.

Beclomethasone Dipropionate and Fluticasone propionate are examples of two highly potent therapeutic agents that are used in extremely low doses. Example 11 details the adoption of the fourth embodiment of this invention to transfer beclomethasone dipropionate from a vapour loaded state to spray dried lactose.

From FIG. 20 it is clear that discrete particles of beclomethasone are deposited on the lactose particles, further, the lactose particles have hairs, have increased surface area, are porous whilst the original particle size is maintained. Further-more the size of the discrete attached beclomethasone dipropionate particles are below two micrometers. The whole particle formulation (i.e. the lactose particles plus adhered beclomethasone dipropoinate particles) is far below 5 micrometers which makes the engineered particles desirable for delivery to the lungs.

Using the above method it is possible to transfer an agent to a particle (host particle) so that the agent forms discrete discontinuous particles, alternatively, the same technique can be used to attach continuous agent particles over the surface of the host particle or even form continuous or discontinuous multilayers. To achieve the latter a long treatment time is required, to alleviate this problems the second aspect of the fourth embodiment is applied.

The application of another aspect of the fourth embodiment is typified by FIG. 16 of example 8 in which the host particles are immersed in the liquid fluid that contains the agent(s) to be transferred to the host particles. From FIG. 16, it is clear that apart from continuously covering the host particle with the transferred agent, the resulting particles have also been architectured to form hairs. Further, the architectured particles have increased in size whilst remaining spheroidal.

It should be obvious to those skilled in the art that more than one agent can be transferred to the host particle and one of such agents transferred may be a constituent of the host particle (see below).

Transfer of an agent (that is a constituent of the host particle) to the host particle using the method of this invention is desirable in that it is safe, fast, economical, controls the shape and the size of the particle whilst repairing surface and crystallographic defects with the option of architecturing (for example forming hairs, pores, hollow volume) in one step. Surface and crystallographic defects such as sites of high energy are reduced, clefts and crevices are filled with the transferred agent and surface irregularities are smoothed, whilst ensuring the content of the host particle is unchanged. High energy sites, clefts, crevices and surface irregularities are known to be the causes of adhesion, cohesion and frictional forces that are the main causes of poor drug delivery to the lung.

The above process is exemplified by example 16, in which lactose was carried in a vapour loaded state and deposited onto microfine lactose.

Accordingly, lactose particles used in conventional dry powder inhalers can be treated by this aspect of the fourth embodiment to decrease the cohesion and adhesion problems normally associated with conventional lactose and especially inhalation grade lactose, other carrier material such as sorbitol, mannitol and the like and also drug particles.

Mass transfer has been done with beclomethasone fluticasone and lactose the former two are water insoluble whereas the latter is water soluble. Hence the mass transfer technique of this fourth embodiment is applicable to water soluble and water insoluble agents to form continuous (fluticasone) and discrete particulates (beclomethasone).

The fifth embodiment of the present invention provides a method of crystallization that not only maintains a spherical shape and high monodispersity of the particles, but also increases the specific surface area of the particles.

Furthermore, it is also clear that hairs are formed on the final particles and the size of the final particles show less polydispersity compared to the starting particles. Since the starting material is glassy and amorphous caused by quench cooling (i.e. extremely rapid cooling) and the final material is crystalline the process of the present invention improves the crystallinity of the final product. The particles produced by the melt back crystallisation technique has passed through at least two changes in states of matter from the frozen state to the liquid state during treatment back to solid state when the final particles are formed (Hence the term MELT-BACK). Thus supporting the claim for changes in states of matter.

The fifth embodiment of the present invention is the application of the method of this invention in a so called “melt back” technique that enables the reduction in the particle size of the starting material by over 1000% if desired without departing from the original shape of the starting particles, reducing cohesion and optionally architecturing (for example forming hairs, pores, hollow volume) the particles. In contrast to this embodiment, traditional micronising techniques such as milling and spray drying that produce highly cohesive, amorphous material. In this current embodiment the reduction in particle size is coupled with an increase in crystallinity and this is in direct contravention to the teachings of the prior art. Particles produced by the fifth embodiment can then be architectured using the other embodiments of this invention.

Essentially this embodiment requires the solidification of a particle, preferably by freezing droplets from solutions, melt, suspensions, emulsion, slurry, paste and the like followed by treating the frozen or non frozen solid particle with a fluid.

The fluid is preferably composed of two miscible mediums containing a dissolved agent (such as polymer). The preferable miscible mediums are ethanol acetone mixture and the preferable agent is the polymer Polyvinylpyrrolidine alcohol.

The above fluid facilitates melting of the frozen particle into a liquid droplet and removal of one or more agents of the liquid droplet such that the liquid droplet reduces in size thereby concentrating the remaining agents in the liquid droplet to exceed the supersaturation point (of the remaining agents in the liquid droplet). This is a starting point from which precipitation or crystallisation proceeds to form a particle whose size is much below that of the starting particle. The final particle is preferably spherical in shape, more preferably spherical and uniform in size and most preferably spherical, uniform in size and having hairs and/or pores.

The application of the fifth embodiment of this invention is shown Example 7.

In example 7 the starting frozen particles are large (in the order of 2-6 mm) yet the final particles are about 30 micrometers, however, using the same syringe with a needle attached to the syringe will produce starting particles which are much smaller than 2-6 mm hence the treated particle will have a size much smaller than 30 micrometers. For those skilled in the art it is clear that techniques for producing very fine liquid droplets, such as atomisation, and such frozen atomised droplets are much smaller than the starting frozen droplets of the above. In this instance nano-sized final particles are envisaged.

### EXAMPLES

### Example 1

The particles to be treated were processed by spray drying. 5 grams (gm) of lactose was dissolved in 100 ml distilled water and the resulting solution was spray-dried using a Buchi 190 mini-spray dryer according to the following conditions:
Inlet temperature: 176° C.,
Outlet temperature: 112° C.,
Aspirator dial reading: 15,
Feed rate: 5 ml/min,

The resulting particles are shown in FIG. 1:

1 gm spray-dried lactose particles were immersed in a 250 ml flat bottom beaker containing 100 ml of hot ethanol (45° C.) (Absolute, 99.7%, BDH, Poole, U.K) for 10 min to form hairy and porous lactose particles. The suspended hairy lactose particles were left to settle and cool for 2 min and were recovered by filtration under vacuum using a Buckner glass funnel. The resulting particles were recovered in a glass petri dish and allowed to dry in a ventilated oven at 50° C. for 16 hours and these particles are shown in FIG. 2:

It will be appreciated that the particles are uniform in size and larger compared to the original spray-dried particles shown in FIG. 1. Compare this Figure to that of FIG. 2.1 which is that of dandelion fluff whose attachments are easily dispersed by a slight wind. It is obvious from FIG. 2 (boxes highlight the detached hairs) that the hairs have detached from the hairy particles (in a similar manner to that of dandelion fluff) and as such it is possible to use the detached hairs alone or in combination. All methods of harvesting the particles using any separation methods known, to those skilled in the art, are embodied within this invention.

Compare FIG. 2 to that of FIG. 2.1; which is a picture of the fruiting head of dandelion fluff. It is clear that there are many similarities between the two. The particles in both are light in density, fluffy and easily carried by and follow the direction of the air-stream. Both have pappus-like projections (hairs), these pappi are easily detachable by a hint of air change and follow the direction of the wind. Both are also spherical. The preparation method was designed to ensure that particles in FIG. 2 are hollow inside which gives them excellent aerodynamic properties making them ideal candidates for inhalation.

The particles of FIG. 2 are hairy and microporous, however, they have tendency to aggregate as shown in FIG. 3.

An additional step, such as, ultrasonication was useful in de-aggregating the agglomerated particles before filtration. From this it is understood, to those skilled in the art, that any de-agglomerating methods to obtain partially or fully de-aggregated particles can be used and are thus embodied within the spirit of this invention. Example 2 is another de-aggregating method, however, in this case the de-aggregation occurs before treatment.

### Example 2

Pretreatment of 10 gm of the original spray-dried lactose (FIG. 1) contained in a rotating metal bowl with ballotoni beads was used to initiate primary hair formation and de-aggregate spray-dried particles. The treating fluid ethanol was introduced as an extremely fine mist to the powder using an air jet nebuliser running at 1 ml/min. 10 ml of treating fluid was nebulised and treated with the powder on successive sequential occasions (total of 50 ml). Intermittent heat by means of hair dryer oriented to the back of rotating bowl was also applied. After every 20 ml of the nebulised treating fluid, hot air was applied for 30 seconds by mean of hair dryer whose airjet was directed to the back of the rotating bowl. This treatment initiated changes in the particle morphological features including nano-hairs and dimple formation (similar to that of a golf ball), which are obvious from FIG. 4 as well as de-aggregating the original spray-dried particles (FIG. 1).

Following pre-treatment, the recovered particles (FIG. 4) were immersed and covered with boiling ethanol for 10 seconds to promote rapid hair formation and morphological changes without destroying the original shape of the particles. These morphological changes include hairs, pores, surface texture, increase in particle size, hollow volume, hair size, pore size, surface area, crystallinity and the like, as well as improved particles flow properties. FIG. 5 shows an example of the particles produced.

It is obvious to those skilled in the art that the particles can be subject to any number of pre-treatments which may include immersion in a medium or subjection to a vapour or any other state of matter and these pre-treatments can be performed in any sequence to obtain particles with other morphological features of which hairs, dimples, pores and the like are included. Further treatments or exposure to vapour can form particles with modified morphological features or enhance the morphological features as shown in the following examples.

The above approach provides an increase in particle size without departing from the original shape, coupled with the presence of hairs on the particle surface and pores. It is evident that there was also an increase in the hollow volume. The particles are composed of 100% of one component (lactose).

### Example 3

5 gm of spray-dried lactose (prepared according to example 1) were treated with 150 ml boiling ethanol contained in a 600 ml flat-bottomed beaker for 60 seconds. The treated particles were recovered by filtration and dried as described in example 1. The resulting particles were stored in a desiccator over silica gel (as desiccant) and the resulting particles are shown in FIG. 6:

FIG. 6 shows hairy and porous lactose particles. These particles have grown in size to about 20 micrometers. Hence there is also an increase in hollow volume. The particles in FIG. 6 present more extensive hair and pore formation compared to those of FIG. 5 suggesting that if the treatment temperature is lower then the treatment time must be greater to obtain a more extensive change in particle morphology. To those skilled in the art both time and temperature need to be manipulated to obtain the desired particle morphology which includes the particle size, surface texture, hairs, pores, hollow volume, cystallinity, particle shape, polymorphic form, surface area, flow properties and the like.

A further example to exemplify this statement is FIG. 7 in which 10 gm spray-dried lactose was immersed in ethanol at ambient temperature for 45 minutes. It is clear that the particles have increased in size to at least 70 micrometers and even though hair projections are formed, the morphology of the hairs and particles are radically different from that of FIGS. 2, 5 and 6. The particles of FIG. 7 may represent the extreme end of hair formation in that the spherical particles lose their shape and tend to form individual single crystals. This is another method of obtaining uniform individual pure crystal without the need of crystallisation from solution, the latter tends to produce crystals of non-uniform size and shape distribution coupled with crystal damage caused by mechanical stirring for example. The particles of FIG. 7 are better able to stabilize the mix due to their extensive projections allowing greater contact area. Such particles can be used to efficiently entrap small drug particles preventing drug detachment during vibration, shipping or handling, thus maintaining a stable uniform mix compared to smooth particles. These projections will also enable de-aggregation in the inhaler device allowing better aerosolisation and dispersion of the particles.

The microbiological analysis of the powders obtained from examples 2 and 3 showed no sign of microbiological contamination. Further, the fluid used (ethanol) is known, from the literature, to have preservative, antiseptic and disinfection properties. Further-more, heated ethanol it known to have sterilization properties.

### Example 4

10 gm of spray-dried lactose prepared in a manner similar to example 1 were introduced in a rotating bowl containing ballotoni beads. The treatment fluid ethanol was introduced as an extremely fine mist to the powder using an air jet nebuliser running at 1 ml/min. 10 ml of treatment fluid was nebulised and treated with the powder on successive sequential occasions (total of 160 ml). Intermittent heat by means of a hair dryer oriented to the back of rotating bowl was also applied. After every 20 ml of the nebulised treatment fluid, hot air was applied for 30 seconds by means of a hair dryer whose airjet was directed to the back of the rotating bowl.

FIGS. 8a, 8b and 8c indicate the changes in particle morphology as increasing amounts of treatment mist were used to architecture the particles.

It is clear from the above Figure that surface texture changes occur with the treatment medium coupled with heat. These changes in the surface texture are seen as surface dimpling and nano-projections.

It is also clear from this figure that longer exposure time to the treatment mist and heat increased the changes in particle morphology such as increased surface roughness, dimpling and projections compared to that of FIG. 8b

It was clear that treatment with vapour is a long process and thus inefficient, advantageously, it does not affect the starting particle size whilst enabling architecturing of the particle surface morphology. Vapour treatment can thus be used for partial particle architecturing or to initiate the particle morphological characteristics so that rapid architecturing can take place in a more efficient manner as obtained with immersion of particles in a liquid. FIG. 9 is a scanning electron micrograph of fully architectured lactose particles immersed in hot ethanol from the starting partially architectured particles as shown in FIG. 8c. The nano-sized hairs of FIG. 8c are grown to micron size hairs of FIG. 9 enabling the operator to control hair size.

Treating with vapour has been shown to improve the flow properties of a powder (example 14) without affecting the particle size. In this present example it is possible not only to architecture the particle whilst maintaining its original particle size it can also be applied to architecturing the particle whilst causing very small or massive changes in the particle size and hairs.

This can be achieved, in the current example, by altering 1) treatment with vapour and applying heat simultaneously, 2) treatment with the vapour first then applying heat at each successive atomization, 3) atomizing the vapour on many successive occasions then applying heat or 4) heating the powder then atomizing the vapour onto the powder. The results suggest that scheme 3 produced much greater changes in particle size, particle morphology and powder properties in a shorter time period compared to the rest. However, the resultant particles can then be immersed in a liquid medium to achieve complete, efficient and uniform architecturing

### Example 5

The following example, wherein, the agents (polyvinyl alcohol and lactose) are both insoluble in the treatment medium.

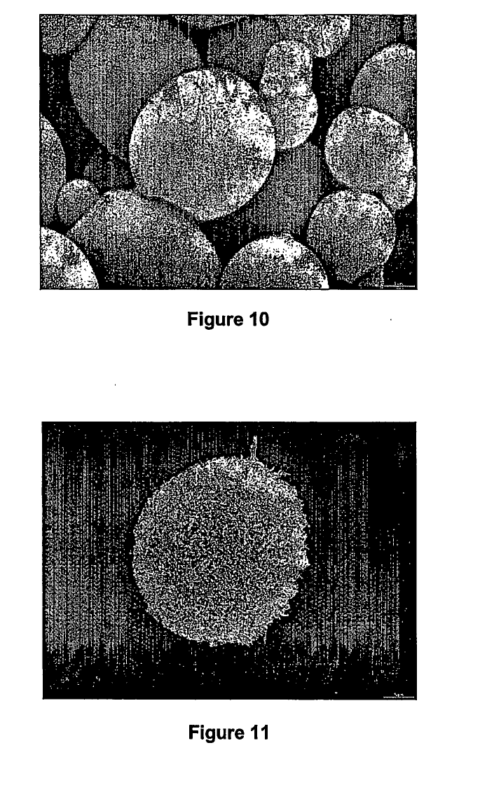

0.1 gm of Polyvinyl alcohol (PVA, 10,000 MW) was dissolved in 100 ml of distilled water at 70° C., once dissolved 5 gm of lactose was dissolved in the PVA solution. The resulting solution was allowed to cool and spray-dried according to the conditions in example 1.1 gm of spray-dried lactose-PVA shown in FIG. 10 was treated with 150 ml boiling ethanol for 30 seconds to form hairy particles as shown in FIG. 11.

Once again, the treated particles are hairy, with many projections and the particles have increased in size.

FIG. 11.1 is a photomicrograph of spray-dried lactose-PVA particles immersed in hot ethanol for 60 seconds.

Longer exposure produces fluffy candyfloss like particles. More extensive hair projections compared to FIG. 11 after a total exposure time of 60 seconds. This example shows that the time of exposure and the temperature of the treatment fluid influence hair architecture. To exemplify the different ways of architecturing hairs compare FIG. 2, FIG. 5, FIG. 6, FIG. 7, FIG. 15 and FIG. 16.

### Example 6

The following example, wherein, the agent (PVP 24000 MW) is soluble in the treatment medium, whereas the agent (lactose) is not soluble in the treatment medium.

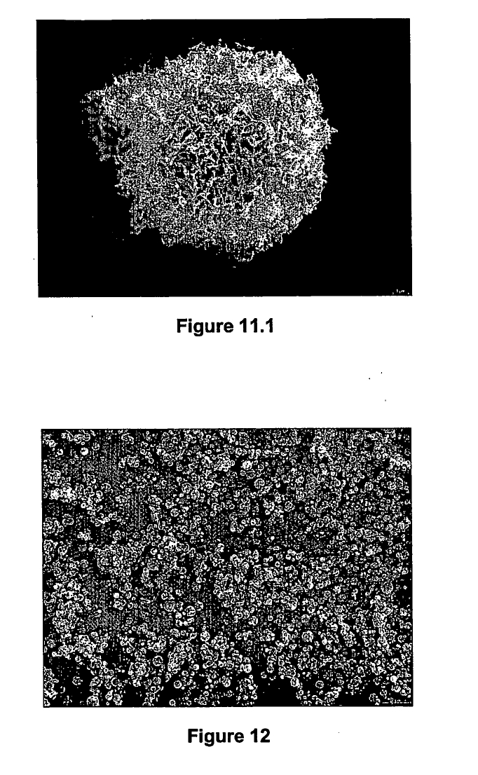

0.025 gm of PVP was dissolved in 100 ml distilled water, 5 gm lactose was dissolved in the PVP solution. The resulting solution was spray-dried according to the procedure detailed in example 1. The spray-dried lactose-PVP particles are shown in FIG. 12. 2 gm of spray-dried lactose-PVP particles were immersed in 150 ml of boiling ethanol for 60 seconds to give particles shown in FIG. 13.

It can be seen that the treated spray-dried lactose-PVP particles still retain their spherical shape, increased in diameter and presented considerable quantities of hairs after conttreatmentacting with ethanol medium.

In the two previous examples PVA and PVP were used one is insoluble, whereas, the other was soluble in the treatment fluid. It is clear that these excipients changed the nature and texture of the hairs and of the particle. So it is possible for the operator to use any agent, treatment fluid or combinations thereof that give particles and hairs of the required nature and texture. The agent(s) can be soluble or insoluble in the treatment fluid.

It is claimed that using this technology, the particle size can be controlled. The previous examples above (example 1 to example 6) showed that particle size could be manipulated to give required particle size. This was achieved by manipulating the operating conditions such as the additives, agent(s), treatment fluid or their combinations. The other morphological features of the particles can be controlled in a similar way to the manner in which the particle size is controlled. For example the polymorphic form of lactose can be controlled by coordinating the use of additives (heat in this example) and treatment fluid to ensure that lactose remained as α-lactose, β-lactose and combinations thereof.

Extension of these principles (including the co-ordination of agents and additives) enables the operator to control the polymorphic form of the agent(s). Since the particles in some of the above examples increased in size, their density was reduced, their hollow volume was increased, their surface area increased, their dissolution rate increased, the particle flow and aerodynamic properties improved. These are some examples, which are not exhaustive, of the way the operator can co-ordinate and manipulate the operating conditions to achieve the required particle characteristics.

### Example 7

In the detailed description solid frozen particles (or frozen droplets) can be architectured by the melt-back procedure, this is an example of this process.

10 gm of lactose was dissolved in 100 ml of distilled water and 20 ml of this solution was introduced drop wise, using a 20 ml syringe, into liquid nitrogen to freeze the droplets. The frozen particles (as shown in FIG. 14) were recovered and introduced in 50 ml of an ethanol/acetone mixture (30/130 v/v) containing 0.025 gm of PVP 24,000 under stirring using Heidolph 4-blade stirrer at 500 rev/min in a 600 ml beaker at ambient temperature. The solvent turned cloudy upon addition of the frozen lactose droplets, however, stirring was continued for 5 minutes. The resulting particles were recovered by filtration, under vacuum. The particles were dried in a ventilated oven at 50° C. for 16 h and stored over silica gel. The particles are shown in FIG. 14.1.

The particles shown in FIG. 14.1 are individual with no signs of agglomeration and the particles are also uniform in size.

The particles of FIG. 14.1 are hairy porous lactose particles. Such particle architecturing is produced by treating the particles with an agent not present in the particles and also treating in a fluid with more than one medium, whilst mechanically stirring the mixture (additive).

The particles are uniform in size which is about 20 micrometers. Smaller particles were obtained using smaller bore syringes or atomizing using a jet nebuliser, air brush, spray gun, spay nozzle and the like.

The particles are spherical as that of the original droplet, the hairs are radically different from that presented in the preceding figures. In this case the hairs are much thicker and extend from the centre of the particle (FIG. 15).

These hairs present themselves as stiff crystalline plate-like blades (FIG. 15.1) compared to the fluffy, and light needled shaped hairs of FIG. 2 and the light, needle like hairs of FIG. 7 and the fluffy, light and candy floss like hairs of FIG. 11.1. In essence this invention allows the user to design the hairs required for the purpose. Furthermore, the hairs can be designed, for example, to be deformable, as shown in FIG. 11.1 and or brittle (as shown in FIG. 15.1). It is understood that these properties are desirable in the pharmaceutical area, for example in inhalation and tabletting as described in the pharmaceutical literature.

In this example as the particle form from the frozen droplet, they are concurrently architectured to form hairs and it is thus an example of architecturing whilst the particle if forming.

### Example 8

A solution of 0.25% w/v of fluticasone in ethanol was prepared by adding 0.25 gm of fluticasone to 100 ml of ethanol in a 600 ml round, flat bottomed flask. The mixture was stirred at 500 rpm at 25° C. until the solution became clear. 10 gm of sprayed-dried lactose, prepared according to the conditions of example 1, was added to the resulting solution The suspension was maintained at a temp of 25° C. and stirred at 500 rpm for 5 minutes using a Heidolph 4 -blade stirrer, which was situated approximately 1 cm above the bottom of the flask. The suspension was filtered and dried according to the conditions in example 1.

It is clear that hairs are formed and the particles have increased in size (to about 20 micrometers) but still retain their spherical shape. This experiment is an example of treating the particle with a fluid in which one of the constituents of the fluid is not present as a constituent of the particle. A bi-layer particle is produced and the coating constituent can act to entrap and retain the activity or protect the base particle from atmospheric effects such as moisture. Extending this technique it is possible to create multi-layer particles whose constituents may or may not be biologically-active. This represents another coating technique, distinct from the traditional methods of coating. In this example a solution is used into which the particles are immersed, equally, the particles may be immersed into a suspension, dispersion, emulsion or the like.

### Example 9

It was claimed that it is possible to architecture the particles as many times as required to obtain particles of the required morphological features. This is an example of this process. 20 gm of vapour-architectured powder according to example 4 was further architectured using 140 ml of ethanol (as a fluid) according to the atomisation protocols of example 4:

The powder was placed in a rotating metal bowl. 10 ml of the fluid was atomised through a nebuliser fitted with a glass connecting tube hanging over the powder bed. During treatment the powder was continuously mixed. After nebulising 10 ml of the fluid, the rotating bowl was heated using a hair drier to evaporate all the fluid. The above process was repeated until the allocated fluid volume had been used. The resulting particles were spread onto a flat drying tray, which was placed in an oven at a temperature of 50° C. for 16 hours. The resulting particles were placed in a desiccator over silica gel.

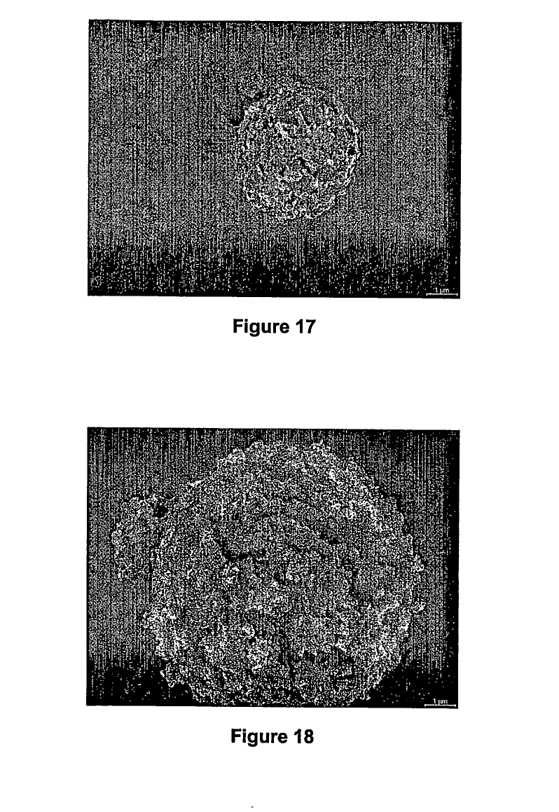

From FIG. 17 it is clear that there has been an increase in the extent of hair formation, compared to FIGS. 8b and 8c, and still the spherical shape whilst the particle size was shifted slightly to the right i.e. a slight increase in particle size (FIG. 25.2). Hence the method of the current invention can shift the whole particle size distribution. It is also clear that increasing the amount of fluid atomized to the particles slowly increases the particle size whilst treating the particles with liquid fluid causes much greater and more rapid changes in particle size and particle features. As a result it may be concluded that treatment of the particles with a vapour is less aggressive than treating the particles with a liquid at equivalent treatment conditions. Apart from the hairs obtained a specific morphological feature, such as the pores in this case, was manipulated in a way so as to increase the extent and number of pores. These characteristics are important in the pharmaceutical and non-pharmaceutical fields. An example in the pharmaceutical field, porous particles have been shown to improve drug delivery to the lung due to their favourable aerodynamic properties (Large porous particles for sustained protection from Carbachol-induced bronchoconstriction in guinea pigs, Abdellaziz Ben-Jebria, et al, Pharm. Res. 16(4):555-561, 1999). Porous particle have desirable properties in that they impart high specific surface area to the powder and thus facilitate improved dissolution rate. In food and cosmetics industries, the porous nature of the particle will retain perfumes, flavourants, fragrances, clothes conditioners, opacifiers, deodorants or any other entrappable constituents for longer periods of time. These particles will maintain and release menthol taste (in smokers tooth paste, chewing gums, gargles), perfume, deodorant effect, release of antiseptics, and release of anti-lice materials to pet coats over a longer time period. In food industry, these particles can be used to entrap multiple flavourants, colourants and fragrances in one particle. These porous and hairy particles are also useful for household products, car industry, pesticides and fertilizer, husbandry industries, tobacco industry, water purification and medical industries

It is possible for the operator to modify or enhance a particular feature of the particle in modifying the operating conditions.

### Example 10

In example 9 the constituents of the particle were insoluble in the fluid, however, in this example the constituents of the particle are soluble in the fluid. Using the same experimental conditions as the example 9, above, the powder from example 9 was further vapour-architectured using 100 ml of a 94/6% ratio of ethanol/water (as the fluid).

It is clear that the use of ethanol/water mixture as a fluid dramatically changes the surface morphology and extent of hair formation. It is possible to choose a pure fluid or a fluid mix that will give the operator particles with the required morphology. It was shown previously that liquid architecturing at elevated temperatures increased the size of the particles. These particles of increased size could then be recovered and vapour-architectured either with pure fluid, fluid mixes or combinations thereof to achieve the required surface and morphological attributes.

In example 9 and example 10 the size, type, nature and number of the pores were manipulated by altering the operating conditions. From FIG. 18 the pores where much larger and different in shape to that of FIG. 17. The importance of the number, shape and size of the pores are that more ingredients (such as flavourants, colourants and drugs) can be incorporated within these particles and their release profiles from these particles can be modified.

### Example 11

Trofast, 1992 (Patent WO 92/18110) used pure anti-solvent vapour, whilst, Trofast, 1995 (Patent WO 95/05805) used pure solvent or anti-solvent vapour to condition a powder in order to make it more stable. This example shows the transfer of a therapeutic agent (beclomethasone (BDP)) onto lactose/PVP particles while architecturing the particles. In this example beclomethasone (BDP) was dissolved in ethanol. The resulting solution was nebulised and used to architecture lactose/PVP particles.

Lactose/PVP spray-dried particles were prepared by dissolving 0.025 g of PVP (24,000 molecular weight) in 100 ml deionised water, to this PVP solution 11.7 gm of lactose was dissolved and the resulting solution was spray-dried using a Buchi spray dryer according to experimental conditions given in example 1.

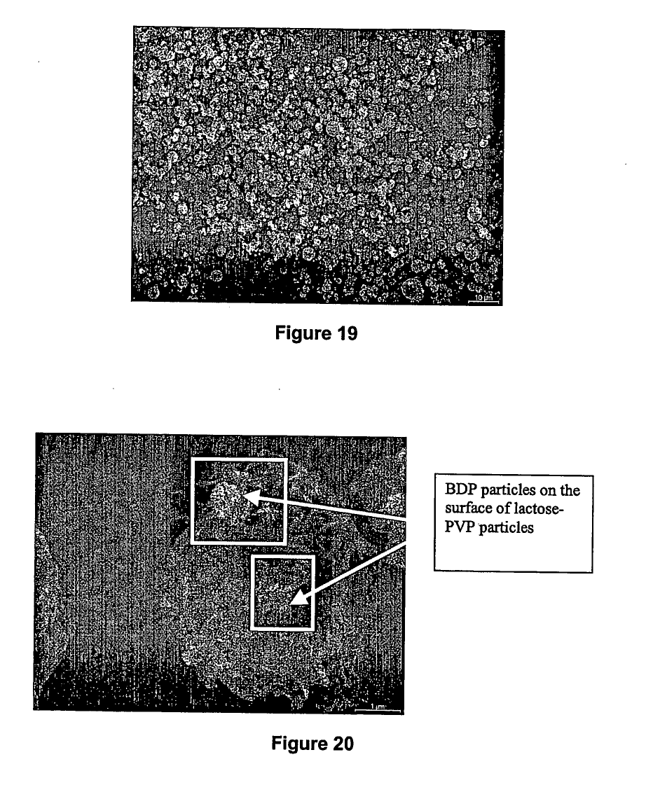

The spray-dried lactose particles in the presence of PVP are shown in FIG. 19.

It is clear from FIG. 19 that the particles are smooth and spherical in shape.

These spray-dried lactose/PVP particles were treated with ethanol vapour containing BDP. The experimental conditions are as follow:

10 mg BDP was dissolved in 50 ml ethanol, the resulting solution was treated with 5 gm of spray-dried particles using an air jet nebuliser running at 1 ml/min. Atomisation was carried out at room temperature. After atomisation the particles were dried at room temperature. The resulting particles are shown in FIG. 20.

The boxed areas on FIG. 20 show the even distribution of BDP particles on the lactose-PVP base particles. PVP was used in this example, as it is known from the previous examples, that it extensively forms hairs in the presence of ethanol. Lactose also forms hairs in the presence of ethanol but to a lesser extent than PVP. Hence the presence of PVP will maximize hair formation whilst minimizing the time of treatment with the fluid, which in this case is ethanol. This example uses two excipients, one of which is soluble (PVP) in the fluid (ethanol), whilst the other (lactose) is insoluble in the fluid (ethanol). The ethanol in the fluid has multiple functionalities, one of which is that it architectures PVP and lactose to form hairs. Secondly, it acts as a carrier for the transfer of an agent (BDP) and to deposit that agent onto the surface of the particles. Ethanol was chosen as it is safe (compared to other solvents), it has high volatility hence it rapidly evaporates leaving the therapeutic agent on the surface of the particle. This shows that the process and materials used are flexibly used to achieve the desired properties for the intended application.

It is obvious from FIG. 20 that the deposited BDP are of suitable size for inhalation purposes. This technology has great applicability in other areas, for brevity, and such example is in tablet formation technology. In tabletting technology it is well known that optimal tablets are obtained when plastically deforming and fragmenting materials are compacted together. The plastically deforming and fragmenting material are usually prepared as a physical mixture, to this physical mixture the drug is added before tabletting. However, in this instance a uniform formulation cannot be assured and de-mixing and segregation always occurs. The particles of the present invention can be successfully used in tabletting as the plastically deforming PVP, fragmenting lactose and drug are incorporated into one particle, assuring formulation uniformity hence better compressibility than physical mixtures, minimizing the processing time, minimizing the cost compared to labour and cost intensive wet and dry granulation techniques routinely used in tablet technology.

It is known that if a potent drug is used in a mix, the amount of drug used is small and to ensure a uniform mix it is necessary to increase the number of drug particles per sample or dose. To do this it is necessary to use a smaller drug particle size, however, producing such very fine powder is difficult and often attended by severe aggregation thus defeating the object of size reduction in the mixing process. When the proportion of active (in this case beclomethasone) is extremely small and finally presented in a small dose unit, physical dry mixing of solids will fail to produce an adequate dispersion of drug within the formulation. To avoid the above drawbacks, the current invention adopts an efficient and reproducible strategy in which the drug is dissolved in ethanol to form a perfect mix. This perfect mix is reduced in size by atomization from an air-jet nebuliser to form a fine mist. This fine mist contains individual liquid droplets whose size is much smaller than that obtainable by conventional milling. Further more these liquid droplets are uniform in size, size distribution and drug content. Hence mixing is carried out by treating lactose-PVP particles contained in a rotating tumbling chamber with atomized liquid droplets. Evaporation of the solvent leaves beclomethasone particles adhered to the surface of the lactose particles. The fine mist surrounds the tumbling lactose-PVP particles resulting in uniform beclomethasone particles on the surface of each lactose-PVP particle (FIG. 20). The solvent also acts as an architecturing agent, in architecturing the lactose and deposited beclomethasone to form particles with desired morphological features, which stabilise the mix hence preventing segregation whilst improving the flow properties of the formulation. This process is rapid and achieves different objectives in one step. These objectives may be; particle size reduction; uniform mixing, architecturing; enhancing the powder flow properties. A suspension, emulsion and the like of the drug can be treated in a similar fashion as discussed above and any particle and any treatment fluid can be used.

### Example 12

Surprisingly, the transfer of agents to the particle (as shown in examples 8 and 11) reduced inter-particulate cohesion and improved the flow of the powder. In both examples, the agent transferred were not part of the particle (hence a bi-constituent particle is obtained) and the transferred agents were hydrophobic. Equally it is also desirable to obtain mono-component particles with low cohesion that produces a powder that flows well. In this example the transferred agent is the constituent agent of the particle (in this case lactose) thus forming a mono-component particle, also this transferred agent is also hydrophilic.

10 gm of lactose was added and dissolved in 100 ml of distilled water. 6 ml of the resulting solution was added to 94 ml of ethanol. 50 ml of this resultant liquid was atomized onto 10 gm of spray-dried lactose (prepared according to example 1) using the atomization protocols described in example 4.

From FIG. 21 it is clear that hairs are formed and the particles are porous and flow tests demonstrated a dramatic improvement in powder flow properties. The mist can also be formed from lactose suspension, emulsion or the like. Pre-treated particles and untreated particles (as shown in example 16) can also be used instead of spray-dried particles in fact particles from any source can be used. Spray-dried lactose was used in this example as spray dried particles are spherical in shape and this spherical shape is a desired property. The processes of this invention have shown that despite the improvement in the powder properties, the original spherical shape was maintained through out. Hence, particles of other shapes can be treated by this invention without altering the shape whilst dramatically improving the particles properties.

### Example 13

Microfine lactose (BDI) as directly obtained from the supplier was treated in three ways;

1) A 100 gm sample was retained in a 45 micrometer mesh size sieve and exposed to liquid nitrogen vapour for about 5 minutes. The resulting powder was spread onto a flat tray and placed, to dry, in a ventilated oven at 50° C. The resulting powder was then stored in a desiccator above silica gel until used.

2) Another 100 gm sample was thinly spread onto a flat stainless steel tray (dimensions 30 cm×23 cm×1.5 cm) and sufficient liquid nitrogen was poured onto the powder to immerse all the powder. The liquid nitrogen was allowed to evaporate at room temperature. To prevent any condensation, the resulting powder was transferred and thinly spread onto another tray of equal dimensions. This latter tray was placed, to dry the powder, in a ventilated oven at 50° C. The resulting powder was then stored in a desiccator above silica gel until used.

3) Another 100 gm sample was treated with liquid nitrogen vapour as described in example 1) above, immediately after exposure to liquid nitrogen vapour, the powder immersed into liquid nitrogen. Again the liquid nitrogen was allowed to evaporate and the resulting powder was transferred and thinly spread onto tray (of the dimensions to that described in 2 above). This latter tray was placed, to dry the powder, in a ventilated oven at 50° C. The resulting powder was then stored in a desiccator above silica gel until used.

All three treatments produced less cohesive and free flowing powder compared to the starting microfine powder. Surprisingly, there was no sign of adhesion of particles to glass containers, in which these treated powders were stored, was observed unlike the untreated powder which aggressively adhered to the glass container wall

The particle size distribution of Microfine lactose and the samples of Microfine lactose prepared according to the above three treatments were determined with a Sympatec Helos Particle Size Analyzer at two dispersion pressures (1 and 3 bar). The results of the analyses are shown from FIGS. 22.2-22.8.

From FIGS. 22.1 to 22.8, at 1 Bar dispersion pressure, untreated Microfine lactose exhibits a broad size distribution with a significant shoulder this shoulder was more apparent using a high dispersion pressure (3 bar). The change in the particle size distribution of untreated Microfine lactose with increased dispersion pressure suggests the presence of substantial particle aggregates, that require high pressure for their de-aggregation and dispersion (See FIG. 24). Whereas, in contrast, All Microfine samples treated with liquid Nitrogen either as a vapour, a liquid or combinations thereof improved and normalised the particle size distribution without affecting the particle size. From the above, a preferred treatment is using the vapour of liquid nitrogen, a more preferred treatment is with liquid nitrogen and the most preferred treatment is with combinations of liquid nitrogen and the vapour of liquid nitrogen. Further, comparing FIGS. 22.4 and 22.8, there is no difference in the particle size distribution even though the dispersion pressure is reduced from 3 bar to 1 bar. This observation is extremely advantageous for dry powder inhalations powders as a low inhalation flow rate is sufficient to disperse particles treated in this manner

Furthermore, this treatment method, in contrast to the prior art, is applicable to water soluble, water insoluble, thermolabile and fragile materials (such as proteins peptides and genes). The method is patient and envornmentally friendly and is easily scalable at minimal costs.

Liquid nitrogen was used for this example, those skilled in the art are aware that other liquefied gases, refrigerants, anaesthetics and other low temperature liquids can be used.

### Example 14

The flow properties of the powder was measured by the angle of repose using the poured method (as described by Wells, J. I., Pharmaceutical preformulation, Ellis Horwood, Chichester, 1988)

| Material | Angle of repose (θ) | |
|---|---|---|
| Spray dried lactose | DOES NOT FLOW | (FIG. 1, from Example 1) |
| Liquid vapour | 28 | (FIG. 8c, from Example 4) |
| introduced to the | | |
| lactose particles | | |
| Lactose particles | 19.15 | (FIG. 6, from Example 3) |
| added to the | | |
| liquid fluid | | |

According to the literature, the lower the angle of repose the better the flowability of the powder and powders with angle of repose less than 30 have good flow, whereas, values below 25 indicate excellent flowability. Thus engineered particles have superior flow properties compared to that of the starting material.

### Example 15

Deposition/Aerodynamic Testing of Engineered and Lactose Crystals

Apparatus:

A modified twin stage impinger apparatus (mTSI) developed for this study (assessing aerodynamic properties of the engineered and non-engineered lactose particles) was based on the standard glass twin stage impinger (Apparatus A of the British Pharmacopeia, BP 2001) (FIG. 23.1). It was employed with a view to deposit lactose and a mixture of lactoses (engineered hairy, engineered porous and non-engineered lactose) onto the adhesive tape placed on an aluminium stub of SEM.

The air jet filter at the lower stage of the standard TSI was removed from the coupling tube and replaced by the microscope stub which was attached to the base of the conical flask by means of blu-tak. A small gap was left between the microscope stub and the coupling tube to enable air flow through the mTSI. 7 ml of distilled water was introduced into the upper chamber of mTSI whilst the lower chamber remained liquid free. Blend were prepared of the following
50 mg spray dried lactose (From example 1, FIG. 1.), 50 mg of hairy lactose (From example 1., FIG. 2) and 50 mg

A glass device with a 29 Quickfit® socket was fitted to the glass throat of the mTSI. to aerosolize 150 mg of hairy lactose particles (of Example 0.1 and FIG. 2) at 60 L/min. This experiment was performed in triplicate using the same flow rate i.e 60 L/min. After each deposition of hairy particles, the mTSI was dismantled and the stub removed from the lower stage flask. The mTSI parts were thoroughly washed and dried between depositions and resultant stubs were viewed using a scanning electron microscope.

From FIG. 23.3 it is evident that engineered hairy lactose particles as large as 60 micrometers deposit in the lower stage of the twin stage impinger. Yet the twin stage impinger at a flow rate of 60 L/min is quoted to have a cut of diameter of between 6.3 to 6.4 micrometer, hence the hairy engineered lactose particles of the present invention must have good aerodynamic properties. Further, from FIGS. 23.3 and 23.4, hairs have detached from the hairy particle during particle flight and upon impact with the stub. Furthermore the brittle nature engineered to the hairy lactose particles have resulted in wholesale fragmentation of hairy lactose particles upon impact with the stub spreading the fragments over a large surface area. The presence of engineered hairy lactose particles on the lower stage of the twin stage impinger suggest that they have a longer time of flight than conventional lactose particles of similar size that are designed to remain in the device or deposit in the upper stage of the twin stage impinger.

### Example 16

0.125 gm of lactose was dissolved in 100 ml of distilled water. 7.5 ml of this lactose solution was added to 292.5 ml of ethanol (purity 99.7% absolute ethanol). The resulting solution was the treatment fluid that was atomized onto microfine lactose, as obtained from the manufacturer.

300 gm of microfine lactose (BDI, U.K) was placed in the bowl of a domestic Platinium Pro Breville mixer. The fluid was introduced as an extremely fine mist to the powder using an air jet nebuliser running at 1 ml/min. 10 ml of fluid was nebulised and used to treat the powder on successive sequential occasions (total of 220 ml). During atomization of the fluid the surface of the particles in contact with the fluid was continuously renewed. After every 20 ml of the nebulised fluid, hot air was applied for 30 seconds by mean of hair dryer whose airjet was directed to the side of the rotating bowl.

The final powder was spread thinly onto a stainless steel tray and placed in an oven maintained at a temperature of 50° C. and the powder allowed to dry for 48 hours. FIG. 0.24. shows the resulting cones formed from the drained method (Aulton, M. E., Pharmaceutics The Science of Dosage form design, Second edition, 2002, page 205).

The amount of lactose carried in the vapour loaded state was chosen to be small so as not to deviate the particle size from that of the starting material. FIG. 24 compares the cones formed, using the drained method, of the untreated microfine lactose and treated microfine lactose. The heap formed by the untreated microfine lactose does not resemble that of a cone (which is should if it has good flow properties) and it exhibits clumped agglomerates that are indicative of cohesion between the particles. Whereas the treated microfine lactose forms a defined smooth heap with no agglomeration or clumps suggesting a reduction in cohesion and consequent improvement in powder flow.

### Example 17

Blend Homogeneity Lactose and Beclomethasone

Measurement of dose uniformity of Beclomethasone Dipropionate from the blend prepared by transferring beclomethasone dipropionate from a vapour loaded state to spray dried lactose.

The homogeneity of the blend was examined by analysing the quantity of BDP in aliquots (400.4±2 mg) of sampled powder, each aliquot of blend was placed in a 100 ml volumetric flask and made up to the volume with HPLC mobile phase (acetonitrile: water in the ratio 70 :30, v/v) and the amount BDP was determined by HPLC (Shimadzu, Japan) using UV detection at 239 nanometers. Ten aliquots were taken randomly from the blend and the resulting solution from each aliquot was assayed in duplicate. The co-efficient of variation (% cv) was used to assess the homogeneity of the blend. The percentage recovery was found to 98.4±2.4 corresponding to a % cv of 2.43. The results suggest a uniform mix was achieved using the mixing procedure described in this embodiment

### Example 18

The particle size distribution of Spray-dried lactose (FIG. 1, Example 1) and the vapour architectured spray dried lactose (Example 9, FIG. 17) determined with a Sympatec Helos Particle Size Analyzer at 1 Bar dispersion pressure. The results of the analyses are shown below.

Note that there was an increase in the volume mean diameter (VMD) from 2.69 μm (for spray-dried before ethanol vapour architecturing) to 7.61 μm (after ethanol vapour architecturing). The particle size distribution has also been shifted towards larger size after treatment with ethanol vapour from example 9.

1-33. (canceled)

34. A particle, having at least one changed morphological, chemical or physical feature, wherein said changed feature can facilitate the attachment of at least one agent to the outer surface of the particle, thus permitting the particle to act as a carrier for said at least one agent;
wherein one changed feature is an increased hollow volume.

35. A particle according to claim 34 wherein the one or more further changed features are selected from the group consisting of hairs, pores, surface dimpling, spongy-like formation, modified particle surface roughness, particle shape, particle size, density, modified specific surface area, reducing cohesiveness, improved powder flow, improvement in aerodynamic properties of the particle, transfer and attachment of at least one agent to the particle, the result of transfer of at least one agent, and combinations thereof.

36. A particle according to claim 34 wherein the particle is spherical in shape.

37. A particle according to claim 34 wherein the particle is between 0.05 μm and 4000 μm in diameter.

38. A particle according to claim 34 wherein the agent is selected from the group consisting of therapeutic agents, prophylactic agents, diagnostic agents, excipients, diluents, flavorants, fragrances, dyes, nutrients, and sweeteners.

39. A particle according to claim 34 wherein the agent is a therapeutic agent selected from the group consisting of corticosteroids, anti-inflammatories, antitussives, bronchodilators, diuretics, anticholinergics, hormones, analgesics, vaginal preparations, antiallergics, anti-infectives, antihistamines, anti-neoplastic agents, anti-tuberculosis agents, proteins, polymeric drugs, lipids, organic substances, inorganic substances, nutrients, pro-drugs, antigens peptides, and derivatives thereof.

40. A particle according to claim 34 wherein the particle is administered by a route selected from the group consisting of pulmonary, oral, parental, nasal, rectal, tonsillar, buccal, intra-ocular, topical/transdermal, and vaginal administration.

41. A particle according to claim 34 wherein the agent is selected from the group consisting of beclomethasone, fluticasone, lactose, polyvinyl pyrrolidone, and polyvinyl alcohol.

42. A particle according to claim 34 wherein the particle itself acts as an agent.

43. A method of treating particles to engineer/architecture the particles with particular chemical, morphological and physical features or combinations thereof, wherein one such feature is an increased hollow volume, said method comprising the steps of
optionally processing, at least one agent to form a particle;
treating the particle by making available a fluid, alone or in combination with at least one additive(s) or further agent(s), to the particle to promote change in one or more of the morphological, chemical or physical features of the particle;
repeating step (b) as many times as necessary;
harvesting engineered particles; and
repeating steps (a) to (d) as many times as necessary.

44. The method of claim 43 wherein a further engineered/architectured feature is the formation of hairs on the surface of the treated particle.

45. A method according to claim 43 wherein the promoted change of step (b) results in at least one further change to the particle, and wherein the further change is selected from the group consisting of forming and or promoting and or controlling the growth of hairs; modifying the properties of the existing hairs; promoting the formation of pores; modifying the properties of existing pores; modifying the density, modifying and controlling the particle size, controlling particle size growth, increasing or decreasing the surface area or specific surface area of the particle; reducing the cohesiveness of the particles; increasing the flow of the powder; forming and/or modifying surface dimpling; formation and/or modification of sponge-like formations; alteration of particle surface roughness, improvement in the aerodynamic properties of the particle, ability of the particles to form a stable uniform mix, and ability of the particles to improve blend uniformity and content uniformity.

46. A method according to claim 43 wherein at least one further agent(s), further fluid(s), further additive(s) or combination thereof, is added to any of stages a) to e).

47. A method according to claim 43 wherein the agent is selected from the group consisting of corticosteroids, anti-inflammatories, antitussives, bronchodilators, diuretics, anticholinergics, hormones, analgesics, vaginal preparations, antiallergics, anti-infectives, antihistamines, anti-neoplastic agents, anti-tuberculosis agents, proteins, polymeric drugs, lipids, organic substances, inorganic substances, nutrients, pro-drugs, antigens peptides and derivatives, and combinations thereof.

48. A method according to claim 43 wherein the agent(s) of the particle is either a combination of polyvinyl alcohol and lactose, a combination of polyvinylpyrolidone and lactose or lactose.

49. A method according to claim 43 wherein the additive is selected from the group consisting of heat, moisture, radiation, pressure, shear forces, magnetic forces, vibration, stirring, vortexing, vacuum, mixing, tumbling, centrifuging, masticating, ultra-sound waves or extruding, electrical, deaggregation agents, and combinations thereof.

50. A method according to claim 43 wherein at least one selected additive is stirring.

51. A method according to claim 43 wherein at least one selected additive is the maintenance of the heat range −200 to 200° C.

52. A method according to claim 43 wherein the engineering step lasts for between 1 microsecond and several hours.

53. A method according to claim 43 wherein the agent of the particle or agent added to the particle during the engineering process is a polymer.

54. A method according to claim 53 wherein the polymer is selected from the group consisting of polyvinyl alcohol, polyvinylpyrolidone and polyethylene glycols.

55. A method according to claim 43 wherein the fluid contains at least one medium, and/or at least one agent, and/or at least one additive and combinations thereof, that promotes changes in any of the morphological, chemical or physical features of the particle.

56. A method according to claim 43 wherein the fluid is in the bulk liquid state, dispersed liquid state, vapor state or combinations thereof and is either aqueous, organic, liquefied gases or a combination thereof.

57. A method according to claim 56 wherein the liquid state is selected from the group consisting of droplets, mist, fog, and spray.

58. A method according to claim 43 wherein the fluid is selected from the group consisting of water, hydrocarbon liquids, halogenated hydrocarbons, mineral spirit, mineral oils, mineral acids, oxygenated solvents, alcohols, nitrogen containing hydrocarbons, sulfur containing hydrocarbons, hetero-atom containing hydrocarbons, anesthetics, liquefied gases such as liquid nitrogen, the vapor from liquid nitrogen or combinations thereof, and refrigerants.

59. A method according to claim 43 wherein the fluid is selected from the group consisting of water, acetone, ethanol, and combinations thereof.

60. A method according to claim 43 wherein engineering the particle with fluid comprises introducing the fluid, which may be static or in motion, to the particle either in bulk, as droplets, as a foam, as a mist, as fog or as a spray.

61. A method according to claim 43 wherein engineering the particle with fluid comprises introducing the particle, which may be in static or in motion, to the fluid either in bulk, as dispersed particles, as droplets, as a foam, as a mist, as fog or as a spray.

62. A method according to claim 43 wherein the further agent added is polyvinylpyrrolidone, lactose or therapeutic agents such as beclomethasone dipropionate or fluticasone propionate.

63. A low density drug carrier particle having hairs on the surface thereof, wherein the particle acts as a carrier for the delivery of either anti-inflammatory drugs, bronchodilator drugs or a combinations thereof into the lungs of a patient via dry powder inhalation.

64. A carrier particle according to claim 63 wherein the drugs being delivered are selected from the group consisting of beclomethasone dipropionate, fluticasone propionate, salbutamol sulfate, and a combination thereof.
